(19) United States
(12) Patent Application Publication
(10) Pub. No.: US 2022/0233542 A1
(43) Pub. Date: Jul. 28, 2022

(54) COMPOSITIONS AND METHODS FOR PREVENTING OR TREATING HUMAN IMMUNODEFICIENCY VIRUS ASSOCIATED PULMONARY ARTERIAL HYPERTENSION

(71) Applicant: University of Georgia Research Foundation, Athens, GA (US)

(72) Inventors: Karen A. NORRIS, Athens, GA (US); Finja SCHWEITZER, Athens, GA (US); Whitney RABACAL, Athens, GA (US)

(73) Assignee: University of Georgia Research Foundation, Athens, GA (US)

(21) Appl. No.: 17/616,502

(22) PCT Filed: Jun. 2, 2020
(86) PCT No.: PCT/US2020/035694

§ 371 Date: Dec. 3, 2021

Related U.S. Application Data
(60) Provisional application No. 62/857,640, filed on Jun. 5, 2019.

(51) Int. Cl. A61K 31/513; A61K 31/40; A61K 31/683; A61K 45/06; A61P 9/12

(52) U.S. Cl. CPC A61K 31/513; A61K 31/40; A61P 9/12; A61K 45/06; A61K 31/683

(57) ABSTRACT

Provided herein are compositions comprising one or more statins and one or more antiretroviral agents, as well as therapeutic or prophylactic treatment methods featuring such combinations. The described compositions and methods are beneficial for treating or preventing human immunodeficiency virus (HIV)-associated pulmonary arterial hypertension (PAH), related conditions, and the symptoms thereof.

### CROSS REFERENCE TO RELATED APPLICATION

This application claims priority to and benefit of Provisional Patent Application No. 62/857,640, filed on Jun. 5, 2019, the entire contents of which are incorporated by reference herein in their entirety.

### STATEMENT OF RIGHTS TO INVENTIONS MADE UNDER FEDERALLY SPONSORED RESEARCH

This invention was made with government support under Grant Nos. 5R01HL131449-04 and 1R01HL131449-01A1 awarded by the National Institutes of Health. The government has certain rights in the invention.

### BACKGROUND

Pulmonary arterial hypertension (PAH) is a life-threatening disease characterized by pulmonary vascular remodeling, elevated pulmonary arterial pressure, and right heart failure. PAH constitutes a subgroup of pulmonary hypertension that includes idiopathic PAH and heritable forms, as well as PAH associated with congenital heart disease, connective tissue disease, portal hypertension, human immunodeficiency virus (HIV), and other infections. PAH occurs in approximately 1 in 200 to 1 in 20 HIV-infected persons, which is 100 to 1000 times greater than the prevalence of PAH in non-HIV infected populations. Despite the improvements in HIV-associated morbidity and mortality, the prevalence of PAH has not changed significantly in the post-ART era. Based on echocardiographic studies, between 15% and 35% of HIV-infected outpatients had elevated pulmonary artery systolic pressures, indicating that PAH may be even more common than previously thought. Even with diagnosis and treatment, prognosis remains poor for both HIV and non-HIV-associated PAH.

In view of the serious nature of PAH and its debilitating effects, particularly in HIV-infected individuals, effective therapeutics and treatment methods are needed to address this serious medical condition.

### SUMMARY OF THE INVENTION

Featured and described herein are compositions and methods for treating or preventing pulmonary arterial hypertension (PAH) associated with an immunodeficiency virus infection (e.g., HIV or SIV). The described methods involve administering one or more stains and one or more antiretroviral agents to a subject in need thereof, in particular, a human subject. In an embodiment, a composition as described herein is administered to a subject in need thereof to treat or prevent PAH, in particular, a human subject infected with HIV or at risk of HIV infection.

In an aspect, a composition for treating or preventing HIV-associated pulmonary arterial hypertension (HIV-PAH) is provided which comprises an effective amount of one or more statins and one or more antiretroviral agents. In an embodiment, the one or more statins is selected from atorvastatin, fluvastatin, lovastatin, pravastatin, rosuvastatin, simvastatin, cerivastatin, pitavastatin, or a combination thereof. In another embodiment, the one or more antiretroviral agents is a nucleoside reverse transcriptase inhibitor, a non-nucleoside reverse transcriptase inhibitor, a protease inhibitor, a fusion or entry inhibitor, an integrase inhibitor, or a combination thereof. In some embodiments, the one or more antiretroviral agents comprises Zidovudine (Retrovir, AZT), Didanosine (Videx, Videx EC, ddI), Stavudine (Zerit, d4T), Lamivudine (Epivir, 3TC), Abacavir (Ziagen, ABC), Tenofovir, (Viread, TDF), Lamivudine, Combivir (combination of zidovudine and lamivudine), Trizivir (combination of zidovudine, lamivudine and abacavir), Emtricitabine (Emtriva, FTC), Truvada (combination of emtricitabine and Tenofovir (tenofovir disoproxil fumarate), Epzicom (combination of abacavir and lamivudine), Nevirapine (Viramune, NVP), Delavirdine (Rescriptor, DLV), Efavirenz (Sustiva or Stocrin, EFV, also part of Atripla), Etravirine (Intelence, ETR), Rilpivirine (Edurant, RPV, also part of Complera or Epivlera), Saquinavir (Invirase, SQV), Indinavir (Crixivan, IDV), Ritonavir (Norvir, RTV), Nelfinavir (Viracept, NFV), Amprenavir (Agenerase, APV), Lopinavir/ritonavir (Kaletra or Aluvia, LPV/RTV), Atazanavir (Reyataz, ATZ), Fosamprenavir (Lexiva, Telzir, FPV), Tipranavir (Aptivus, TPV), Darunavir (Prezista, DRV), Enfuvirtide (Fuzeon, ENF, T-20) Maraviroc (Selzentry or Celsentri, MVC), Raltegravir (Isentress, RAL), Elvitegravir (EVG, part of the combination Stribild), Dolutegravir (Tivicay, DTG), or a combination thereof. In an embodiment, the one or more antiretroviral agents is emtricitabine and tenofovir disoproxil fumarate. In some embodiments, the statin is atorvastatin and the one or more antiretroviral agents is emtricitabine and tenofovir disoproxil fumarate. In some embodiments, the composition includes a pharmaceutically acceptable carrier, excipient, diluent, or vehicle. In some embodiments, the composition prevents or reduces the severity of HIV-PAH and/or at least one symptom associated with HIV-PAH. In some embodiments, the composition prevents or reduces mean pulmonary arterial pressure. In some embodiments, the composition reduces or prevents pulmonary vascular collagen deposition or right ventricular collagen deposition. In some embodiments, the composition prevents the onset of HIV-PAH. In some embodiments, the composition increases the level of IL-15 in blood or plasma of a subject having HIV-PAH.

Another aspect of the present disclosure provides a method of treating or preventing pulmonary arterial hypertension (PAH) in a subject infected with HIV or at risk of being infected by HIV in which the method involves administering an effective amount of one or more statins and one or more antiretroviral agents to the subject.

Another aspect of the present disclosure provides a method of preventing HIV-associated pulmonary arterial hypertension (HIV-PAH) in a subject having a propensity to develop HIV-PAH, in which the method involves administering to a subject in need thereof an effective amount of one or more statins and one or more antiretroviral agents.

Another aspect of the present disclosure provides a method for treating or preventing pulmonary vascular collagen deposition or right ventricular collagen deposition in an HIV-infected subject or in a subject at risk of HIV infection, in which the method involves administering to a subject in need thereof an effective amount of one or more statins and one or more antiretroviral agents.

In another aspect, a method of increasing the levels of IL-15 in blood or plasma of an HIV-infected subject or in a subject at risk of HIV infection is provided, in which the method involves administering to a subject in need thereof an effective amount of one or more statins and one or more antiretroviral agents. In accordance with the method, upregulation of IL-15 levels is associated with protection from PAH.

In various embodiment of the methods of any of the above-delineated aspects, the subject is at risk of being infected by HIV. In another embodiment, the subject is infected with HIV. In another embodiment, the one or more statins and the one or more antiretroviral agents are co-administered to the subject. In another embodiment, the one or more statins and the one or more antiretroviral agents are co-administered to the subject simultaneously or concurrently. In another embodiment, the one or more statins is administered at a predetermined time before or after the one or more antiretroviral agents is administered to the subject. In an embodiment, the one or more statins is administered prior to infection or post-acute phase of infection to decrease the incidence of PAH. In another embodiment, the one or more statins and the one or more antiretroviral agents are administered to the subject within twenty-four hours of each other. In an embodiment, the one or more statins and the one or more antiretroviral agents are present in a pharmaceutical composition. In some embodiments, the one or more statins is selected from atorvastatin, fluvastatin, lovastatin, pravastatin, rosuvastatin, simvastatin, cerivastatin, pitavastatin, or a combination thereof. In a particular embodiment, the one or more statins is atorvastatin. In some embodiments, the one or more antiretroviral agents comprises a nucleoside reverse transcriptase inhibitor, a non-nucleoside reverse transcriptase inhibitor, a protease inhibitor, a fusion or entry inhibitor, an integrase inhibitor, or a combination thereof. In some embodiments, the one or more antiretroviral agents comprises Zidovudine (Retrovir, AZT), Didanosine (Videx, Videx EC, ddI), Stavudine (Zerit, d4T), Lamivudine (Epivir, 3TC), Abacavir (Ziagen, ABC), Tenofovir, (Viread, TDF), Lamivudine, Combivir (combination of zidovudine and lamivudine), Trizivir (combination of zidovudine, lamivudine and abacavir), Emtricitabine (Emtriva, FTC), Truvada (combination of emtricitabine and Tenofovir (tenofovir disoproxil fumarate), Epzicom (combination of abacavir and lamivudine), Nevirapine (Viramune, NVP), Delavirdine (Rescriptor, DLV), Efavirenz (Sustiva or Stocrin, EFV, also part of Atripla), Etravirine (Intelence, ETR), Rilpivirine (Edurant, RPV, also part of Complera or Epivlera), Saquinavir (Invirase, SQV), Indinavir (Crixivan, IDV), Ritonavir (Norvir, RTV), Nelfinavir (Viracept, NFV), Amprenavir (Agenerase, APV), Lopinavir/ritonavir (Kaletra or Aluvia, LPV/RTV), Atazanavir (Reyataz, ATZ), Fosamprenavir (Lexiva, Telzir, FPV), Tipranavir (Aptivus, TPV), Darunavir (Prezista, DRV), Enfuvirtide (Fuzeon, ENF, T-20) Maraviroc (Selzentry or Celsentri, MVC), Raltegravir (Isentress, RAL), Elvitegravir (EVG, part of the combination Stribild), Dolutegravir (Tivicay, DTG), or a combination thereof.

In another aspect, a method of treating or preventing pulmonary arterial hypertension (PAH) in a subject infected with HIV or at risk of being infected by HIV is provided in which the method involves administering to the subject an effective amount of the above-described compositions.

In another aspect, a method of preventing HIV-associated pulmonary arterial hypertension (HIV-PAH) in a subject having a propensity to develop HIV-PAH is provided in which the method involves administering to a subject in need thereof an effective amount of the above-described compositions.

In another aspect, a method of preventing HIV-associated pulmonary arterial hypertension (HIV-PAH) in a subject diagnosed with HIV-PAH is provided in which the method involves administering to a subject in need thereof an effective amount of the above-described compositions at the time that the subject is diagnosed with HIV.

In some embodiments of the methods of any of the above-delineated aspects, the subject is a human subject. In some cases, the subject is a human patient with HIV. In some embodiments, the subject is a human patient treated at the time of diagnosis of HIV infection. In some embodiments, the subject is a human patient treated at the time of diagnosis of HIV infection and during the course of HIV infection. In some embodiments, the one or more statins or the composition is administered to a subject in need thereof prior to HIV infection or post-acute phase of infection.

In still another aspect, a pharmaceutical pack is provided, which includes one or more statins and one or more antiretroviral agents, wherein the one or more statins and the one or more antiretroviral agents are formulated together or separately and in individual dosage amounts. In some embodiments, the one or more statins and the one or more antiretroviral agents are formulated together. In some embodiments, the one or more statins is selected from atorvastatin, fluvastatin, lovastatin, pravastatin, rosuvastatin, simvastatin, cerivastatin, pitavastatin, or a combination thereof. In a particular embodiment, the one or more statins is atorvastatin. In some embodiments, the one or more antiretroviral agents is a nucleoside reverse transcriptase inhibitor, a non-nucleoside reverse transcriptase inhibitor, a protease inhibitor, a fusion or entry inhibitor, an integrase inhibitor, or a combination thereof. In some embodiments, the one or more antiretroviral agents comprises Zidovudine (Retrovir, AZT), Didanosine (Videx, Videx EC, ddI), Stavudine (Zerit, d4T), Lamivudine (Epivir, 3TC), Abacavir (Ziagen, ABC), Tenofovir, (Viread, TDF), Lamivudine, Combivir (combination of zidovudine and lamivudine), Trizivir (combination of zidovudine, lamivudine and abacavir), Emtricitabine (Emtriva, FTC), Truvada (combination of emtricitabine and Tenofovir (tenofovir disoproxil fumarate), Epzicom (combination of abacavir and lamivudine), Nevirapine (Viramune, NVP), Delavirdine (Rescriptor, DLV), Efavirenz (Sustiva or Stocrin, EFV, also part of Atripla), Etravirine (Intelence, ETR), Rilpivirine (Edurant, RPV, also part of Complera or Epivlera), Saquinavir (Invirase, SQV), Indinavir (Crixivan, IDV), Ritonavir (Norvir, RTV), Nelfinavir (Viracept, NFV), Amprenavir (Agenerase, APV), Lopinavir/ritonavir (Kaletra or Aluvia, LPV/RTV), Atazanavir (Reyataz, ATZ), Fosamprenavir (Lexiva, Telzir, FPV), Tipranavir (Aptivus, TPV), Darunavir (Prezista, DRV), Enfuvirtide (Fuzeon, ENF, T-20) Maraviroc (Selzentry or Celsentri, MVC), Raltegravir (Isentress, RAL), Elvitegravir (EVG, part of the combination Stribild), Dolutegravir (Tivicay, DTG), or a combination thereof. In a particular embodiment, the one or more antiretroviral agents comprises emtricitabine and tenofovir disoproxil fumarate.

Other features and advantages of the invention will be apparent from the detailed description, and from the claims.

### BRIEF DESCRIPTION OF THE DRAWINGS

FIG. 1 is a graph comparing the pulmonary artery systolic pressures of HIV-infected and HIV-uninfected individuals.

FIG. 2 is a set of images illustrating the morphological changes in tissues obtained from normal and SIV-infected rhesus macaques.

FIG. 3 is a diagram illustrating the characteristics of acute and chronic SIV infection in rhesus macaques, and the parameters measured in test subjects, e.g., CD4⁺ T cell count, viral load, peripheral blood mononuclear cell (PBMC)/bronchoalveolar lavage (BAL) cell analysis, inflammatory mediators, pathology and hemodynamic function.

FIGS. 4A-4D illustrate physiological characteristics of SIV-infected rhesus macaques at 0 months post infection (mpi), at 6 mpi, and at the terminal stage of the disease. FIG. 4A is a graph illustrating increased right atrial (RA) pressure in infected animals after infection at the different stages. FIG. 4B is a graph illustrating increased right ventricular systolic pressure (RVSP) in infected animals after infection at the different stages. FIG. 4C is a graph illustrating increased mean pulmonary arterial pressure (mPAP) in infected animals after infection at different stages. FIG. 4D is a graph of systemic and diastolic blood pressure in infected animals at 0 mpi, 6 mpi, and at the end of the study.

FIG. 5 is a graph comparing mPAP in rhesus macaques at baseline (i.e., before PAH onset) and in rhesus macaques with (PH+) and without (PH−) PAH.

FIG. 6 is a series of graphs demonstrating the non-progressive, progressive, and transient PAH phenotypes based on observed mPAP levels in SIV-infected macaques at different timepoints during infection.

FIGS. 7A and 7B demonstrate the non-progressive, progressive, and transient PAH phenotypes based on the level of CD4⁺ T cells and viral load present in SIV-infected macaques at different timepoints during infection. FIG. 7A is a graph illustrating the level of CD4⁺ T cells in SIV-infected macaques with non-progressive, progressive, and transient PAH phenotypes. FIG. 7 B is a graph illustrating the viral load present in SIV-infected macaques with non-progressive, progressive, and transient PAH phenotypes.

FIGS. 8A-8F illustrate collagen phenotypes observed in PAH⁺ and PAH macaques. FIG. 8A is a graph showing a higher percentage of collagen in the right ventricle (RV) compared to the left ventricle (LV) of SIV-infected animals with PAH. FIG. 8B is a graph illustrating that higher collagen percentages are observed in RVs in PAH− and PAH+ macaques compared to the percent collagen in LVs in PAH- and PAH⁺ macaques. FIG. 8C is a graph illustrating a higher percent of collagen in macaques having progressive and transient PAH (P-PAH and T-PAH, respectively) compared to PAH⁻ macaques. FIG. 8D is a graph illustrating a correlation between collagen percentage in the right ventricle and mPAP in SIV-infected macaques. FIG. 8E is an image of tissue from the RV of a macaque without PAH (PAH). FIG. 8F is an image of tissue from the RV of a macaque with PAH (PAH⁺).

FIGS. 9A-9C illustrate a partial cytokine profile associated with PAH in SIV-infected animals. FIG. 9A is a graph showing that levels of IL-15 cytokine are decreased in in plasma of PAH⁺ macaques, including progressive and transient PAH (P-PAH⁺ and T-PAH⁺, respectively) compared to plasma IL-15 levels in PAH⁻ macaques. FIG. 9B is a graph showing an inverse relationship between mPAP at 6 mpi and plasma IL-15 levels. FIG. 9C is a graph illustrating a positive correlation between P-PAH⁺ and T-PAH⁺ and plasma macrophage inflammatory protein 1α (MIP1α) levels.

FIGS. 10A-10E show increased RV glucose uptake in PAH⁺ macaques as measured by ¹⁸FDG PET-CT. FIG. 10A is a representative image of FDG-PET/CT overlay in ventricles of a PAH⁺ animal). FIG. 10B is a representative PET image of ventricles in a PAH⁻ animal. FIG. 10C is a represent PET image of PAH⁺ animal. In FIGS. 10A-10C, the arrows denote the animal's right ventricle (RV). FIG. 10D is a graph illustrating the elevated RV/LV standard uptake value (RV/LV SUV) ratios in SIV-infected macaques (terminal, n=6) compared to uninfected (naïve) macaques (n=11). FIG. 10E is a graph illustrating the relationship of peak mPAP and RV/LV SUV in PAH⁺ macaques (n=6).

FIGS. 11A-11D present timelines and graphs illustrating that statin treatment prevents hemodynamic alterations associated with SIV infection in a NHP model of HIV-PAH. FIG. 11A is a schematic timeline of statin treatment, simian immunodeficiency virus (SIV) infection, and serial right heart catheterization (RHC) of three animal (SIV-infected macaques) study groups/cohorts. FIG. 11B presents graphs comparing mean pulmonary arterial pressure (mPAP) among the study cohorts (either SIV-infected, statin-untreated, PAH− or PAH⁺ animals (Group 1) or SIV-infected, statin-treated animals (Groups 2 and 3) at baseline, 6 months post-infection (mpi), and terminal timepoints. FIG. 11C presents graphs showing mPAP in the statin untreated and treated SIV-infected animal cohorts, as calculated from right ventricular systolic pressure (RVSP), where mPAP (tn mmHg)=0.65×(RVSP)+0.55. PAH is defined as mPAP≥25 mm Hg as indicated by the dashed lines. FIG. 11D presents graphs showing RVSP used to calculate mPAP in animals of the Groups 1, 2 and 3 cohorts. For FIGS. 11B-11D, serial and group characterizations of hemodynamics were analyzed using repeated measures mixed modeling, and the data presented represent the mean±standard deviation (SD).

FIGS. 12A and 12B demonstrate that statin treatment does not significantly affect peripheral blood CD4⁺ T cells or viral load throughout SIV infection. FIG. 12A is a graph comparing CD4⁺ T cells of cohort animals in SIV/Untreated Group 1, SIV/Statin Group 2, and SIV/Statin Group 3 versus time post-infection. FIG. 12B is a graph illustrating viral load in SIV/Untreated Group 1, SIV/Statin Group 2, and SIV/Statin Group 3 animals as analyzed by multiple unpaired t-test analysis using the using the Holm-Sidak method, with α=0.05. Data represent the mean±SD.

FIGS. 13A-13E show that statin treatment prevents SIV-PAH associated changes in plasma IL-15 and BALF TGF-β at 6 mpi and at terminal plasma levels of MIP-1α and TNFα. FIG. 13A is a graph showing bronchoalveolar lavage fluid (BALF) TGF-β levels in the SIV-infected, statin untreated or statin-treated animal cohorts at 6 mpi. FIG. 13B is a graph showing plasma levels of MIP-1α in the SIV-infected, statin untreated or statin-treated animal cohorts at time of terminal infection. FIG. 13C is a graph illustrating terminal plasma levels of TNF-α in the SIV-infected, statin untreated or statin-treated animal cohorts at time of terminal infection. FIG. 13D is graph illustrating plasma IL-15 levels in the SIV-infected, statin untreated or statin-treated animal cohorts at 6 mpi. For FIGS. 13A-13D, the Mann-Whitney U test was used for statistical analysis. Data represent the mean±SD. FIG. 13E shows graphs illustrating Spearman correlation analysis between mPAP (mm Hg) and plasma IL-15 (pg/mL) at 6 mpi in SIV/untreated animals (Group 1, leftmost graph) and SIV/statin-treated cohort Groups 2 and 3 (center and right graphs). In these figures, “R” denotes the Spearman coefficient.

FIGS. 14A-14J demonstrate that statin treatment prevents SIV-PAH associated changes in peripheral blood CD14^{dim}CD16⁺ non-classical monocytes and BALF CD14⁺ CCR7⁻CD163⁻CD206⁺ macrophages at 6 mpi. FIG. 14A is graph illustrating the absolute cell numbers of CD14^{dim}CD16⁺ non-classical monocytes in the peripheral blood of SIV-infected animals in the Group 1-3 cohorts as shown. FIG. 14B is a series of graphs summarizing a correlation analysis between mPAP and CD14^{dim}CD16⁺ non-classical monocytes in SIV/untreated animals (left) and statin-treated cohorts (center and right). FIG. 14C is a graph illustrating the absolute cell numbers of CD14⁺ CCR7⁻CD163⁻CD206⁺ macrophages in the BALF obtained from cohort animals. FIG. 14D is a series of graphs summarizing a correlation analysis between mPAP and CD14⁺ CCR7⁻CD163⁻CD206⁺ BALF macrophages in SIV/untreated animals (left) and statin-treated cohorts (center, right). For FIGS. 14A and 14C, the Mann-Whitney U test was used for statistical analysis. Data represents the mean±SD. For FIGS. 14B and 14D, Spearman correlation analysis was used. FIG. 14E is a graph showing the number of CD4⁺CD16⁻ cells/μl observed in HIV-infected rhesus macaques before infection, 6 mpi, and at the end of the study. FIG. 14F is a graph comparing the number of CD4⁺CD16⁻ cells/μl observed in PAH⁺ and PAH⁻ subjects. FIG. 14G is a graph showing the number of CD4⁺CD16⁻ cells/μl at increasing RVSP values at 6 mpi. FIG. 14H is a graph showing the number of CD4⁻CD16⁺ cells/μl observed in HIV-infected rhesus macaques before infection, 6 mpi, and at the end of the study. FIG. 14I is a graph comparing the number of CD4⁻CD16⁺ cells/μl observed in PAH⁺ and PAH⁻ subjects. FIG. 14J is a graph showing the number of CD4⁻CD16⁺ cells/μl at increasing RVSP values at 6 mpi.

FIGS. 15A-15E present graphs demonstrating that statin treatment prevents SIV-PAH and SIV-associated collagen deposition in the heart and pulmonary arteries. FIG. 15A is a plot illustrating right ventricle (RV) collagen deposition quantified from Masson's trichrome stained heart sections obtained from SIV-PAH⁺ and SIV-PAH⁻ untreated cohorts (Group 1) and from SIV-PAH⁺ cohorts (Groups 2 and 3) receiving statin treatment. FIG. 15B shows a correlation analysis between peak mPAP and % RV Collagen/170 mm² in SIV/untreated animals (Group 1, leftmost graph) and statin-treated animals (Group 2, center, and Group 3, right). FIG. 15C is a representative set of images of Masson's trichrome stained right ventricle sections obtained from untreated SIV-infected animals (Group 1) and SIV-infected animals treated with statin (Groups 2 and 3). FIG. 15D is a plot illustrating pulmonary periarteriolar collagen deposition quantified from Picro-Sirius Red stained lung sections obtained from untreated SIV-infected animals (Group 1) and SIV-infected animals treated with statin (Groups 2 and 3). FIG. 15E presents graphs showing a correlation analysis between peak mPAP and % Area threshold in untreated SIV-infected animals (Group 1, left) and SIV-infected animals treated with statin (Groups 2 and 3, center, right, respectively). For FIGS. 15A and 15D, the Mann-Whitney U test was used for statistical analysis. Data represent the mean±SD. For FIGS. 15B and 15E, Spearman correlation was used.

### DETAILED DESCRIPTION

### Definitions

By “ameliorate” is meant to decrease, reduce, suppress, attenuate, diminish, arrest, or stabilize the development or progression of a disease or disorder.

An antiviral agent refers to a compound or drug that treats infection (or disease) caused by a virus. In particular, an antiretroviral agent is a compound (e.g., a small molecule compound) or drug that treats infection (or disease) caused by an RNA virus or retrovirus, such as immunodeficiency virus (HIV) or simian immunodeficiency virus (SIV), which infects non-human primates.

By “antiviral composition,” “antiretroviral composition,” “antiviral therapy,” or “antiretroviral therapy (ART)” is meant a therapeutic composition or therapy for the treatment of a viral or a retroviral infection (e.g., SIV or HIV). An antiretroviral composition or therapy can comprise one or more antiretroviral agents, compounds, or drugs for suppressing a retrovirus, for stopping or slowing the growth or progression of the retrovirus, and/or for inhibiting the transmission of the virus, for example, by disrupting, blocking, or otherwise interfering with viral replication or propagation following virus infection of a cell. In an embodiment, an antiretroviral composition includes one or more, or a combination of, antiretroviral agents (drugs) that treat infection and disease caused by a retrovirus, such as immunodeficiency virus (HIV) or simian immunodeficiency virus (SIV) that infects non-human primates, such as Rhesus macaques. By way of nonlimiting example, antiretroviral agents or drugs include nucleoside reverse transcriptase inhibitors or nucleoside analog reverse transcriptase inhibitors (NRTIs), e.g., Zidovudine (Retrovir, AZT), Didanosine (Videx, Videx EC, ddI), Stavudine (Zerit, d4T), Lamivudine (Epivir, 3TC), Abacavir (Ziagen, ABC), Tenofovir, a nucleotide analog (Viread, TDF), Lamivudine, Combivir (combination of zidovudine and lamivudine), Trizivir (combination of zidovudine, lamivudine and abacavir), Emtricitabine (Emtriva, FTC), Truvada (combination of emtricitabine and tenofovir (tenofovir disoproxil fumarate)), and Epzicom (combination of abacavir and lamivudine)); non-nucleoside reverse transcriptase inhibitors (e.g., Nevirapine (Viramune, NVP), Delavirdine (Rescriptor, DLV), Efavirenz (Sustiva or Stocrin, EFV, also part of Atripla), Etravirine (Intelence, ETR), and Rilpivirine (Edurant, RPV, also part of Complera or Epivlera)); protease inhibitors (e.g., Saquinavir (Invirase, SQV), Indinavir (Crixivan, IDV), Ritonavir (Norvir, RTV), Nelfinavir (Viracept, NFV), Amprenavir (Agenerase, APV), Lopinavir/ritonavir (Kaletra or Aluvia, LPV/RTV), Atazanavir (Reyataz, ATZ), Fosamprenavir (Lexiva, Telzir, FPV), Tipranavir (Aptivus, TPV), and Darunavir (Prezista, DRV)); virus entry or fusion inhibitors (e.g., Enfuvirtide (Fuzeon, ENF, T-20) and Maraviroc (Selzentry or Celsentri, MVC)); and HIV integrase inhibitors (e.g., Raltegravir (Isentress, RAL), Elvitegravir (EVG, part of the combination Stribild), Dolutegravir (Tivicay, DTG)). An antiretroviral composition or therapy may include additional compounds or drugs.

A “statin” generally refers to any of a group of drugs (compounds) that inhibit the synthesis of cholesterol and promote the production of low density lipoprotein (LDL)-binding receptors in the liver, thus resulting in a decrease in the level of LDL and frequently an increase in high density lipoprotein (HDL) circulating in the blood/plasma. More specifically, a statin refers to a class of compounds comprising 3-hydroxy-3-methylglutaryl coenzyme A (HMG-CoA) reductase inhibitors. Examples of statins include, but are not limited to atorvastatin, fluvastatin, lovastatin, pravastatin, rosuvastatin, simvastatin, cerivastatin, and pitavastatin. Statins may have lipid-independent effects, e.g., anti-inflammatory effects (e.g., decrease CRP, decrease chemokine release (MCP-1, RANTES), decrease adhesion molecules (e.g., P-selectin, VLA-4, CD11a, CD11b, CD18) and decrease cytokine expression (e.g., IL-1ß, TNF-α, IL-6 and IL-8)); immunomodulatory effects (e.g., decrease MHC-II expression and activity, decrease TLR-4, decrease T-cell activation via LFA-1 blockade, decrease proliferation of monocytes and macrophages); effects on smooth muscle and endothelial cell function (e.g., attenuate proliferation and decrease migration of smooth muscle cells and increase eNOS and superoxide scavenging of endothelial cells); and antioxidant effects (e.g., decrease NADPH oxidase and increase Haem oxygenase activation).

Nonlimiting examples of statins include atorvastatin (Lipitor), fluvastatin (Lescol, Lescol XL), lovastatin (Mevacor, Altoprev), pravastatin (Pravachol), rosuvastatin (Creston; simvastatin (Zocor) and pitavastatin (Livalo).

“Atorvastatin” or “Lipitor” refers to a statin having the following structure:

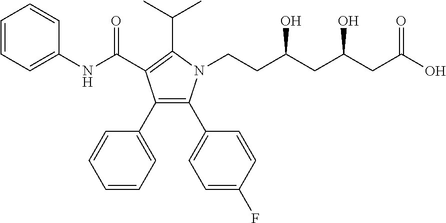 [SMILES: CC(C)c1c(C(=O)Nc2ccccc2)c(-c2ccccc2)c(-c2ccc(F)cc2)n1CC[C@@H](O)C[C@@H](O)CC(=O)O]

“Cerivastatin” or “Baycol” or “Lipobay” refers to a statin having the following structure:

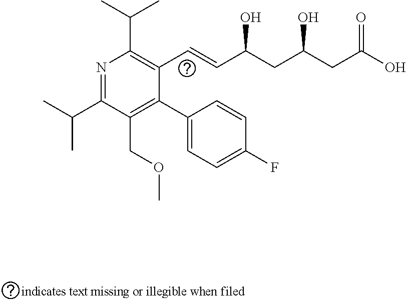 [SMILES: COCc1c(C(C)C)nc(C(C)C)c(/C=C/[C@@H](O)C[C@@H](O)CC(=O)O)c1-c1ccc(F)cc1]

“Fluvastatin” or “Lescol” refers to a statin having the following structure:

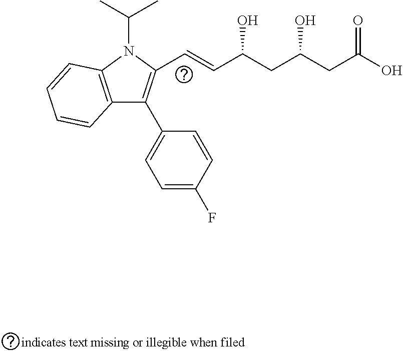 [SMILES: CC(C)n1c(/C=C/[C@H](O)C[C@H](O)CC(=O)O)c(-c2ccc(F)cc2)c2ccccc21]

“Lovastatin,” “Mevacor,” or “Altocor” refers to a statin having the following structure:

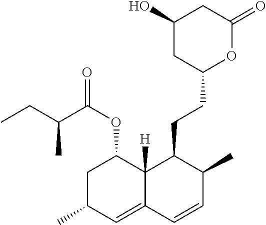 [SMILES: CC[C@H](C)C(=O)O[C@H]1C[C@@H](C)C=C2C=C[C@H](C)[C@H](CC[C@@H]3C[C@@H](O)CC(=O)O3)[C@H]21]

“Mevastatin” or “Compactin” refers to a statin having the following structure:

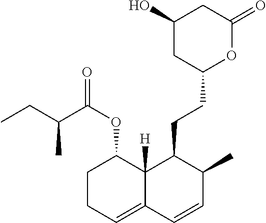 [SMILES: CC[C@H](C)C(=O)O[C@H]1CCC=C2C=C[C@H](C)[C@H](CC[C@@H]3C[C@@H](O)CC(=O)O3)[C@H]21]

“Pitavastatin” or “Livalo” refers to a statin having the following structure:

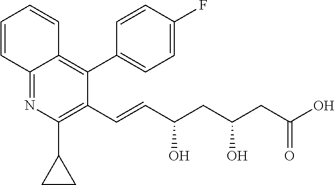 [SMILES: O=C(O)C[C@H](O)C[C@H](O)/C=C/c1c(C2CC2)nc2ccccc2c1-c1ccc(F)cc1]

“Pravastatin” or “Pravachol” refers a statin having the following structure:

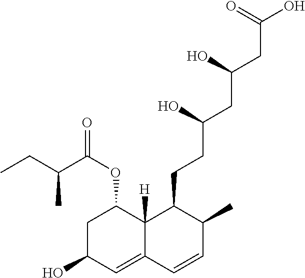 [SMILES: CC[C@H](C)C(=O)O[C@H]1C[C@H](O)C=C2C=C[C@H](C)[C@H](CC[C@@H](O)C[C@@H](O)CC(=O)O)[C@H]21]

“Rosuvastatin” or “Crestor” refers to a statin having the following structure:

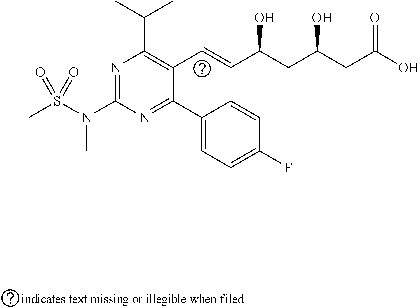 [SMILES: CC(C)c1nc(N(C)S(C)(=O)=O)nc(-c2ccc(F)cc2)c1/C=C/[C@@H](O)C[C@@H](O)CC(=O)O]

“Simvastatin,” “FloLipid” and “Zocor” refers to a statin having the following structure:

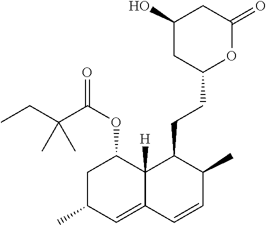 [SMILES: CCC(C)(C)C(=O)O[C@H]1C[C@@H](C)C=C2C=C[C@H](C)[C@H](CC[C@@H]3C[C@@H](O)CC(=O)O3)[C@H]21]

In an embodiment, a composition as described herein may comprise one or more antiretroviral agents and one or more additional compounds or drugs. In an embodiment, a composition as described herein comprises one or more antiretroviral drugs and one or more statins. In an embodiment, the composition is a pharmaceutically acceptable composition which comprises a pharmaceutically acceptable diluent, excipient, carrier, or vehicle. In a particular embodiment, a composition described herein comprises a statin, e.g., atorvastatin, and one or more antiretroviral agent or a combination of antiretroviral agents, e.g., emtricitabine and tenofovir, e.g., tenofovir disoproxil fumarate (Truvada), and a pharmaceutically or physiologically acceptable diluent, excipient, carrier, or vehicle.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “an effective amount” is meant the amount of a compound or composition required to ameliorate a disease or the symptoms of a disease, such as pulmonary arterial hypertension (PAH), in particular, HIV-associated PAH. In an embodiment, an effective amount of the compound or composition prevents the onset of PAH. In an embodiment, an effective amount of the compound or composition or treats a subject having PAH, in particular, an HIV-infected patient having PAH.

By “isolated” is meant free to varying degrees from components which normally accompany it as found in its native state. In various embodiments, an isolated compound is at least 90%, 95%, 96%, 97%, 98%, 99% or even 100% pure.

By “prevent,” “preventing,” “prevention,” “prophylactic treatment” is meant reducing the probability or risk of developing a disease, disorder, or condition in a subject, who does not have, but is at risk of or susceptible to, developing a disease, disorder, or condition.

“Pulmonary arterial hypertension” or “PAH” is a disease that affects the arteries of the lungs. Pulmonary arteries are blood vessels that carry blood from the right side of the heart through the lungs. PAH refers to hereditary and idiopathic conditions characterized by high blood pressure in the lungs, pulmonary vascular remodeling, and right heart failure. In PAH, increased pressure in the blood vessels is caused by obstruction in the small arteries in the lung (pulmonary arteries). A hemodynamic definition of PAH is a mean pulmonary artery pressure (mPAP) of at least 25 mm Hg with at pulmonary capillary wedge pressure less than or equal to 15 mm Hg. In PAH, the pressures in the right side of the heart (right ventricle (RV)) and the pulmonary arteries are elevated, while the pressures in the left side of the heart (left ventricle (LV)) are normal.

By “treat,” treating,” “treatment,” and the like refer to reducing or ameliorating a disease or disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

The therapeutic methods described herein (which include prophylactic treatment) in general comprise administration of a therapeutically effective amount of the compounds or compositions herein, such as a compound or composition described herein, to a subject (e.g., animal, human) in need thereof, including a mammal, particularly a human. Such treatment will be suitably administered to subjects, particularly humans, suffering from, having, susceptible to, or at risk of a disease, disorder, or symptom thereof. Determination of those subjects “at risk” can be made by any objective or subjective determination by a diagnostic test (e.g., genetic test, enzyme or protein marker, family history, and the like) or opinion of a subject or health care provider. The compounds and compositions as described herein may be also used in the treatment of any other disorders in which a viral or retroviral infection and PAH may be implicated.

### Human Immunodeficiency Virus-Associated Pulmonary Hypertension (HIV-PAH)

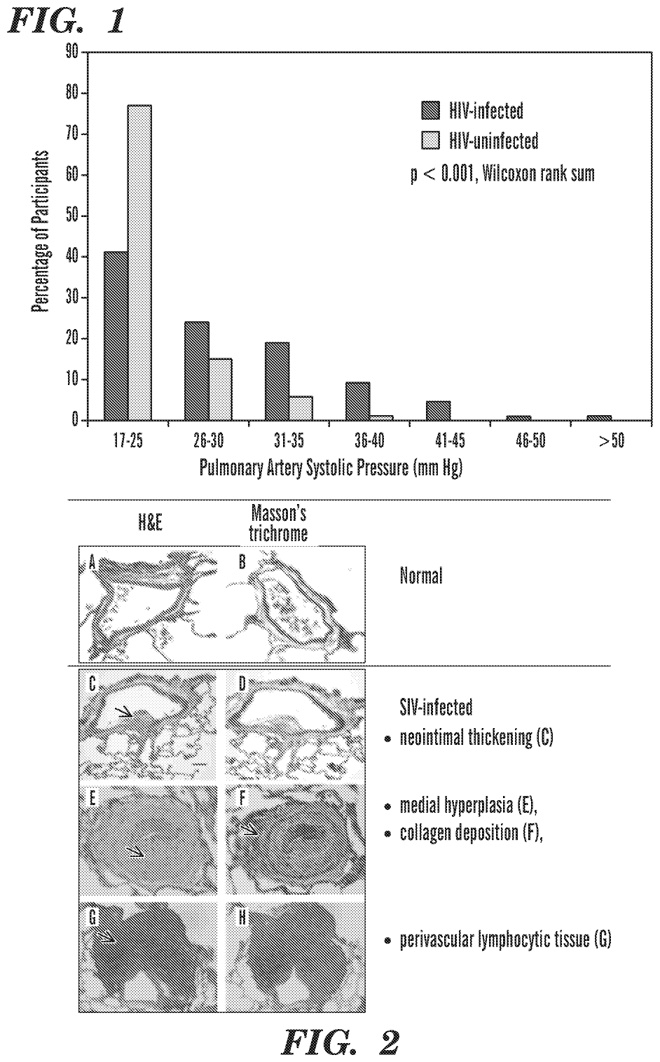

HIV infection increases risks for the progressive disease pulmonary arterial hypertension (PAH). HIV-associated PAH or HIV-PAH incidence is estimated to be 0.5-5% based on pathology and catherization studies. It has been observed that HIV-infected patients have a higher prevalence of echocardiographic abnormalities, such as elevated pulmonary artery systolic pressure (PASP) and tricuspid regurgitant velocity (TRV) (FIG. 1, Hsue et al., AIDS. 2008 Apr. 23; 22(7):825-33)). For example, 35% of HIV-infected patients in one study were found to have elevated PASP of 30 mm Hg compared to just 7.7% in uninfected individuals (p=0.001), and 6.6% of the HIV infected patients had elevated PASP above 40 mm Hg. In a cross-sectional study of 116 HIV-infected outpatients evaluated using Doppler-echocardiography and pulmonary function testing, 64% of the patients had elevated PASP of greater than 30 mm Hg and 15.5% had elevated PASP above 40 mm Hg. Additionally, 7.8% had TRV of 3 m/s and this metric was significantly associated with CD4 cell counts below 200 cells/ml and higher log HIV-RNA levels. Forced expiratory volume in 1 s (FEV1) percentage predicted, FEV1/forced vital capacity, and diffusing capacity for carbon monoxide (DLco) percentage predicted were significantly lower in those with elevated PASP or TRV. (Morris et al., AIDS. 2012 Mar. 27; 26(6):731-40).

Previous studies involving SIV-infected rhesus macaques provide insight into viral-mediated PAH in primates, including humans. Nonhuman primates (NHPs) such as rhesus macaques share many physiological traits with their human relatives including their hemodynamic profiles (Table 1). In addition, pulmonary vascular tissues are remodeled in SIV-infected macaques (FIG. 2).

TABLE 1
Hemodynamics in Humans and NHPs

| Normal Hemodynamic | *Human | Rh Macaque (Mean, +/− |
|---|---|---|
| Values | (mm Hg) | SD (mm Hg) |
| Right Atrial Pressure | 0-6 | 3.09, +/− 0.3 |
| Right Ventricular Systolic | 15-30 | 27.13, +/− 1.2 |
| Pressures | | |
| Mean Pulmonary Artery | 9-19 | 15.42, +/− 0.6 |
| Pressure | | |
| Systolic Blood Pressure | <120 | 119.8, +/− 3.2 |
| Diastolic Blood Pressure | <80 | 70, +/− 2.28 |

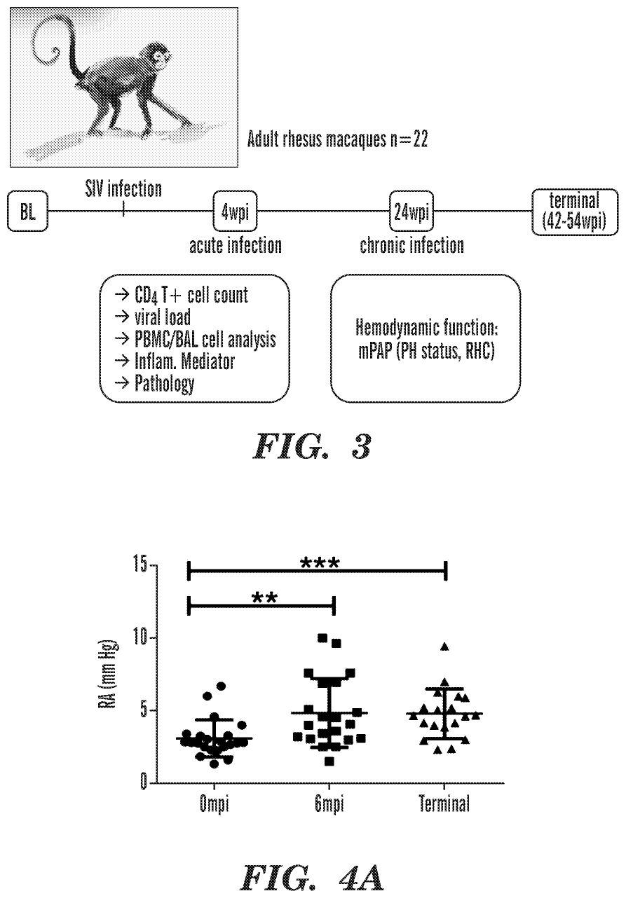

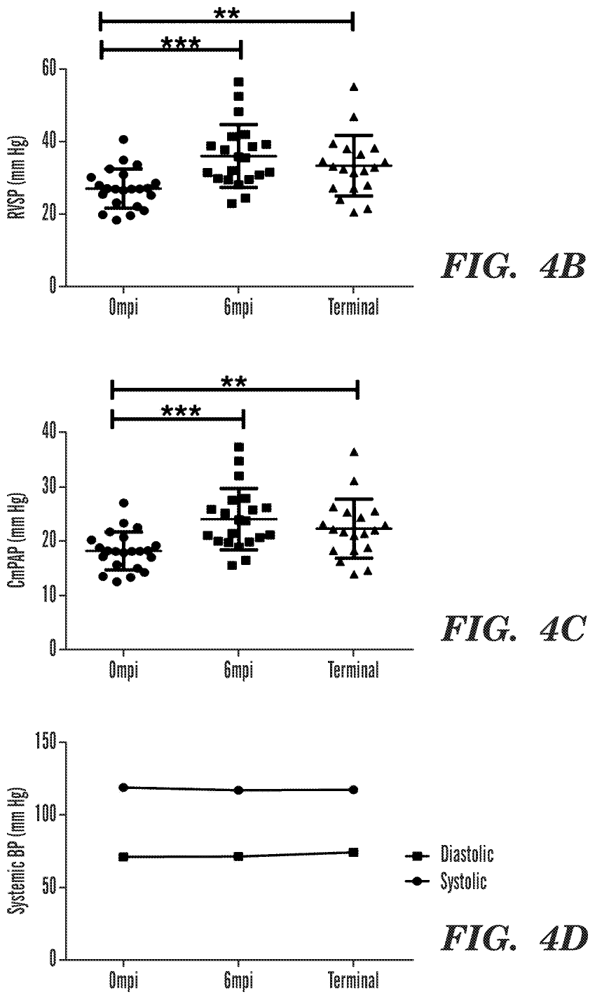

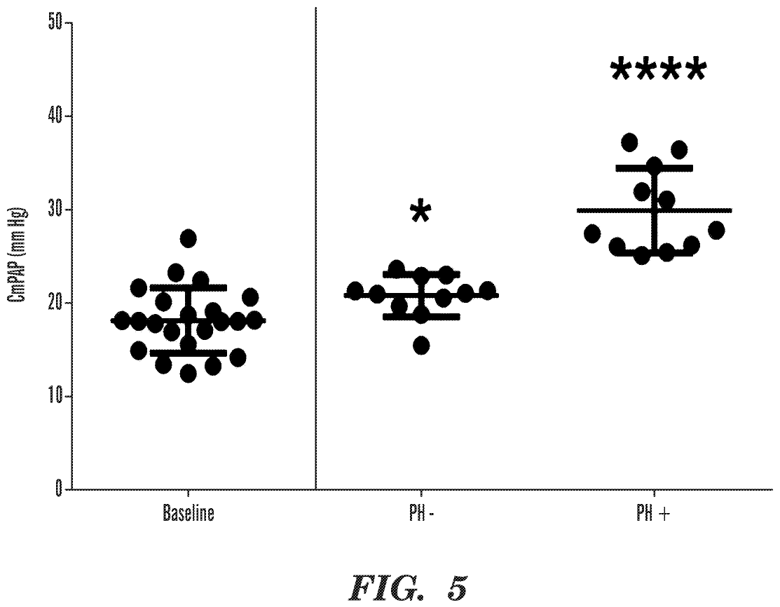

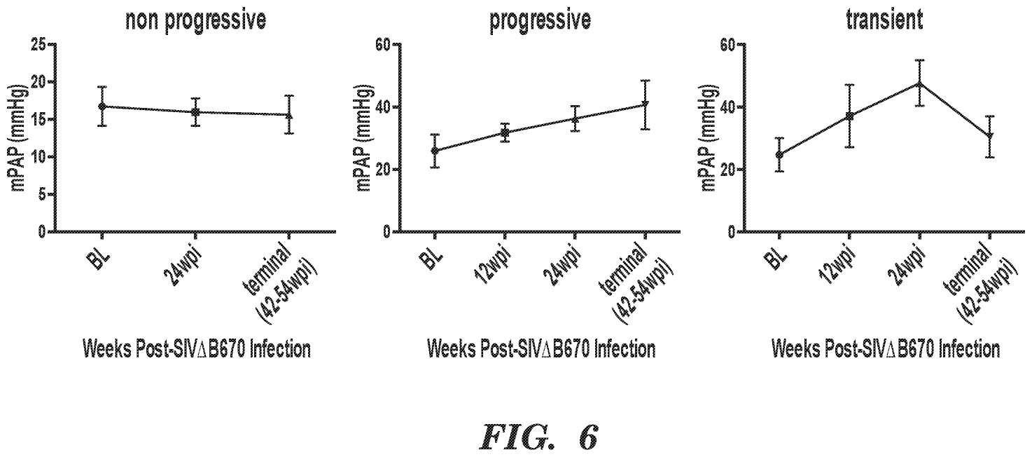

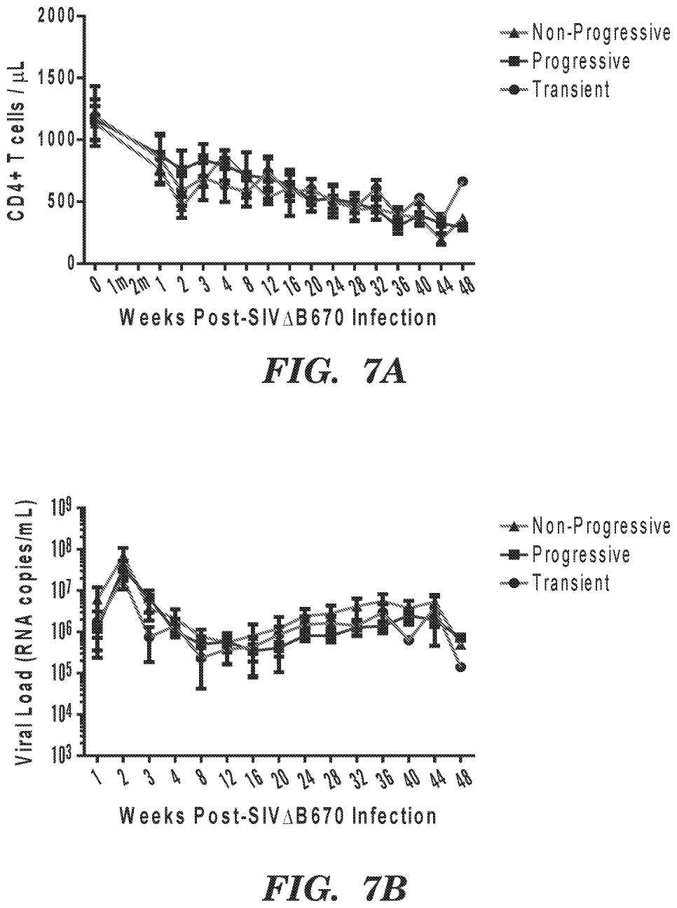

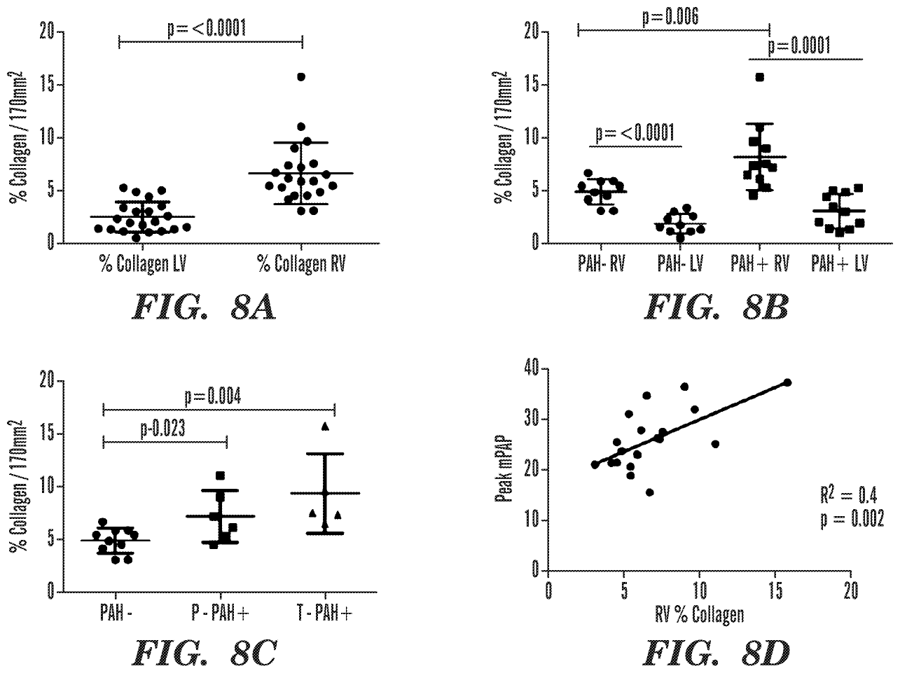

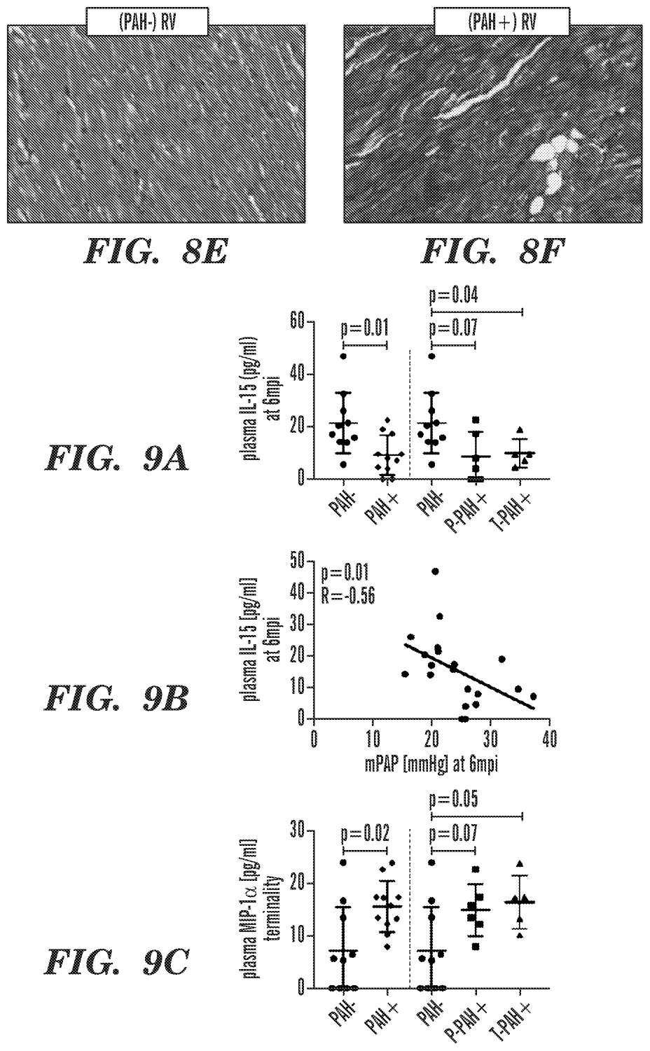

Physiological changes accompanying SIV infection were studied using the timeline shown in FIG. 3. In general, 4 weeks post infection (wpi) is referred to as the acute infection phase, 24 wpi is referred to as the chronic infection phase, and the terminal phase occurs between 42 and 45 wpi. Longitudinal hemodynamics in the infected macaques showed that SIV infection of the NHPs was associated with increased right atrial (RA) pressure, right ventricular systolic pressure (RVSP), and mean pulmonary arterial pressures (mPAP) (FIGS. 4A-4D; FIG. 5). (Tarantelli, et al., 2018, Comp. Med., Vol. 68, No. 6, pages 461-473, the contents of which are incorporated herein by reference). Subsets of the macaques with SIV displayed non-progressive, progressive, or transient PAH (FIG. 6), and CD4 T cell levels and viral loads did not differ between these three PAH phenotypes (FIGS. 7A and 7B). Moreover, pulmonary vascular collagen deposition is increased in SIV-PAH. Additionally, Collagen deposition is increased in the right ventricles of macaques with SIV-PAH⁺ compared to PAH⁻ macaques (FIGS. 8A-8F).

PAH⁺ animals had increased frequency of pro-inflammatory, non-classical monocytes (CD14^{dim}CD16⁺) and increased frequency of pro-inflammatory, alveolar macrophages CD14⁺ CCR7⁻CD163⁻CD206⁺ in BALF macrophages (e.g., M1 and M2 macrophages). Increased frequencies of pro-inflammatory, non-classical monocyte and macrophage phenotypes correlated with elevated RVSP (p=0.04; p=0.03). PAH⁺ animals showed frequencies of tissue resident inflammatory M1-like CD68⁺STAT1⁺ (p=0.001) and M2a-like CD68⁺STAT3⁺ macrophages (p=0.003) and a lower frequency of anti-inflammatory M2c-like CD68⁺STAT6⁺ macrophages (p=0.003) as well as fewer IL-10⁺ cells (p=0.01). (Schweitzer, F. et al. Monocyte and Alveolar Macrophage Skewing Is Associated with the Development of Pulmonary Arterial Hypertension in a Primate Model of HIV Infection. AIDS Res Hum Retroviruses 35, 63-74 (2019), the contents of which are incorporated herein by reference).

Plasma IL-15 levels were decreased in PAH⁺ NHPs, and these levels were negatively correlated with mean pulmonary arterial pressure (mPAP) (FIGS. 9A, 9B). Conversely, plasma levels of MIP1a were increased in PAH⁺ subjects compared to PAH (FIG. 9C).

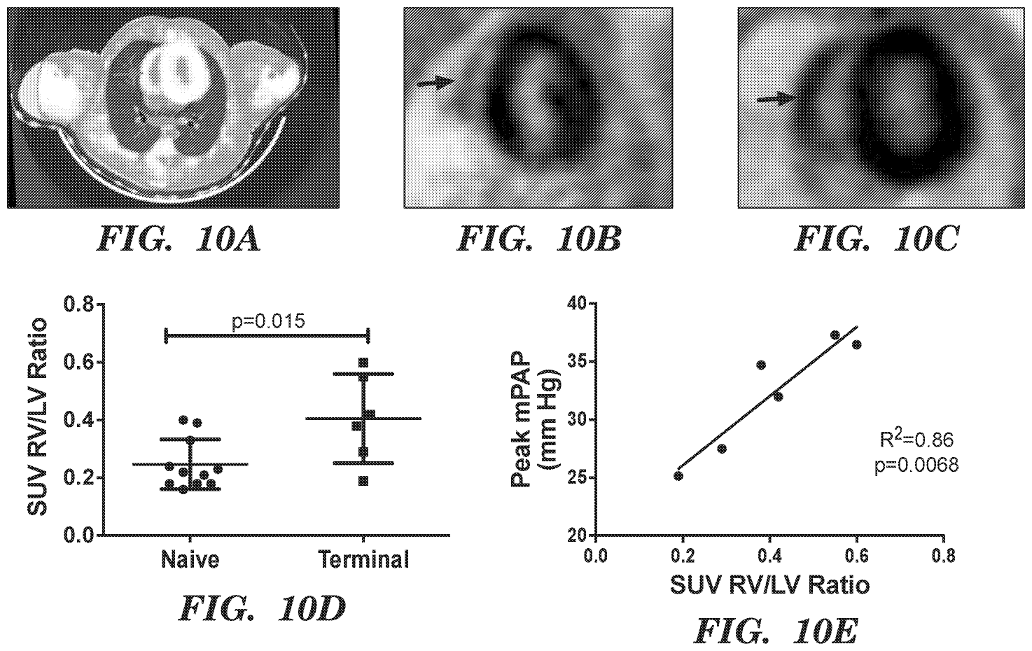

Glucose uptake by the ventricles can be measured using Positron emission tomography with 2-deoxy-2-[fluorine-18]fluoro-D-glucose integrated with computed tomography (¹⁸F-FDG PET/CT) (See FIGS. 10A-10C). SIV infected macaques also exhibited elevated standard uptake values for the right ventricle compared to left ventricle, and the ratio of these values was shown to have a linear relationship with peak mPAP (FIGS. 10D, 10E).

Dysregulation of vascular stiffness and cellular metabolism occurs early in pulmonary hypertension (PH) and stiffening of the vascular extracellular matrix (ECM) results in mechano-activation of the transcriptional coactivators Yes-associated protein 1 (YAP) and TAZ (or WWRT1). YAP/TAZ activation modulates metabolic enzymes, including glutaminase (GLS1), to coordinate glutaminolysis and glycolysis. Glutaminolysis, an anaplerotic pathway, replenishes aspartate for anabolic biosynthesis, which is critical for sustaining proliferation and migration within stiff ECM. Glutaminase (GLS1) and YAP1 are upregulated in pulmonary vasculature in SIV-PAH Macaques. Right ventricle (RV) fibrotic processes and metabolic (e.g., glycolytic) shift occur in early stage SIV-PAH, and ECM stiffening sustains vascular cell growth and migration through YAP/TAZ-dependent glutaminolysis, thereby linking mechanical stimuli to dysregulated vascular metabolism.

The compositions and methods provided herein relate to the discovery as described herein that the administration of one or more statins combined with one or more antiretroviral agents, or an antiretroviral therapy, ameliorate symptoms, treat, or prevent the occurrence or progression of pulmonary arterial hypertension (PAH) associated with infection of a subject with an immunodeficiency retrovirus, such as simian immunodeficiency virus (SIV) or human immunodeficiency virus (HIV). Without wishing to be bound by theory, the one or more statins provide anti-inflammatory therapeutic effects, in combination with the one or more antiretroviral drugs, to achieve the amelioration and treatment of PAH in HIV (or SIV)-infected subjects, including humans.

### Anti-Retroviral and Statin Combination Therapies

As described herein, one or more antiretroviral agents (e.g., drugs or compounds), an antiretroviral drug combination, or an antiretroviral therapy (ART) is combined or co-formulated with one or more statins for the treatment and prevention of immunodeficiency virus-associated pulmonary arterial hypertension (HIV-PAH). In some embodiments, the immunodeficiency virus is simian immunodeficiency virus (SIV) or human immunodeficiency virus (HIV). In some embodiments, one or more antiretroviral drugs or the antiretroviral therapy comprises a nucleoside reverse transcriptase inhibitor, a non-nucleoside reverse transcriptase inhibitor, a protease inhibitor, a fusion or entry inhibitor, or an integrase inhibitor, or a combination thereof (e.g., an “antiretroviral cocktail”). In some embodiments, the antiretroviral therapy is a combination of emtricitabine, tenofovir disoproxil fumarate (e.g., Truvada) as shown in Table 2. As is appreciated by the skilled practitioner in the art, Truvada may be provided as a prophylactic (PrEP) treatment (prior to the onset of HIV) and as a therapeutic treatment (following and during infection with HIV). In some embodiments, emtricitabine and tenofovir disoproxil fumarate can be administered with an additional antiretroviral therapy. In an embodiment, the combination therapy is administered to a subject prophylactically to prevent onset of HIV-PAH. In an embodiment, the combination therapy is administered to a subject as a treatment for HIV-PAH. In an embodiment, the subject is a human patient who is at high risk for HIV and/or HIV-PAH.

TABLE 2

| | | Treat HIV-1 | |
|---|---|---|---|
| Truvada for | | infection | |
| PrEP, once daily, | | in people | |
| orally (PrEP | | who weigh | |
| denotes pre- | | at least 35 kg | In a 35-kg |
| exposure) | Drug Type | (77 pounds) | person |
| Emtricitabine | nucleoside analogue | 200 mg | 5.7 mg/kg |
| | reverse transcriptase | | |
| | inhibitor (NRTI) | | |
| tenofovir | nucleoside analogue | 300 mg | 5.6 mg/kg |
| disoproxil | reverse transcriptase | | |
| fumarate | inhibitor (NRTI) | | |

| Additional Drugs | | | |
|---|---|---|---|
| used for PrEP | | | In a 35-kg |
| with Truvada | Drug Type | | person |
| Raltegravir | Integrase inhibitor | 400 mg, orally, | 11.4 mg/kg |
| (Isentress) | | twice daily | |

In some embodiments, the combination therapy comprises a statin selected from atorvastatin (Lipitor), fluvastatin (Lescol), lovastatin (Mevacor or Altoprev), pravastatin (Pravachol), rosuvastatin (Crestor), simvastatin (FloLipid and Zocor), cerivastatin (Baycol or Lipobay), mevastatin (Compactin or Mevinolin), pitavastatin (Livalo), or a combination thereof.

As described herein, a composition is provided which comprises a combination of at least one statin and one or more antiretroviral drugs for the treatment of HIV-PAH. In an embodiment, the composition comprises one or more statins and one or more antiretroviral drugs or a combination of antiretroviral drugs. In an embodiment, the composition comprises a statin, emtricitabine and tenofovir. In an embodiment, the composition comprises atorvastatin, emtricitabine and tenofovir. In an embodiment, the composition comprises atorvastatin and the combination of emtricitabine and tenofovir disoproxil fumarate (Truvada). In an embodiment of any of the foregoing, the composition is a pharmaceutically acceptable composition which comprises a pharmaceutically acceptable carrier, excipient, diluent, or vehicle.

As further described herein, the combination therapy comprises a method of treating a subject having or at risk of developing, PAH and its symptoms, in which the method comprises administering to the subject an effective amount of a pharmaceutical composition as described above. In an embodiment, the method involves administering the pharmaceutical composition to the subject prior to the subject having or developing HIV-associated PAH, prior to the subject having or being diagnosed as having HIV infection, or prior to post-acute phase of infection. In another embodiment, the method involves administering the pharmaceutical composition to the subject at the time that the subject is diagnosed as being infected with HIV. In another embodiment, the method involves administering the pharmaceutical composition to the subject at the time that the subject is diagnosed as being infected with HIV as well as thereafter, e.g., during and throughout the course of HIV infection. In another embodiment, the method involves administering the pharmaceutical composition to the subject after the subject is diagnosed as being infected with HIV, as well as during and throughout the course of HIV infection. The combination therapy allows for preventing the onset or development of PAH and its symptoms, or reducing the severity of PAH and its symptoms. In a particular embodiment, the composition comprises atorvastatin and Truvada. In embodiments, the subject is a human patient who is not infected with HIV or the subject is a human patient who is infected with HIV.

As described herein, a combination therapy comprises a method of treating a subject having or at risk of developing, PAH, in which the method comprises administering to the subject an effective amount of one or more statins and an effective amount of one or more antiretroviral drugs or a combination of antiretroviral drugs. In embodiments, the statin and the one or more antiretroviral drugs or the antiretroviral drug combination may be administered concurrently; or the statin may be administered at a predetermined time before or after the administration of the antiretroviral drug or drug combination. In an embodiment, one or more statins is administered in conjunction with the one or more antiretroviral drugs or a combination of antiretroviral drugs (e.g., antiretroviral therapy).

The combination of one or more antiretroviral drugs and a statin (or one or more statins) may have synergistic activity, such that the effect of the combination is more than the expected additive effects of the individual components. In addition, compositions that include metabolites of the antiretroviral therapy, statins, and any derivatives thereof are contemplated. Many of these metabolites share one or more biological activities with the parent compound and, accordingly, can also be used in an antiretroviral therapy/anti-PAH combination as. The use of such combinations for the manufacture of a medicament for the treatment or prevention of PAH, namely, SIV- or HIV-associated PAH, is further provided.

In some embodiments, the combination therapy compositions and methods as described herein increase the levels of the IL-15 cytokine in the blood or plasma of an HIV-infected patient or an HIV-PAH patient.

### Methods of the Invention

The methods and compositions provided herein can be used to treat or prevent the onset or progression of HIV-associated PAH and its symptoms. Provided are therapeutic combinations comprising effective amounts of one or more antiretroviral drugs or a combination of antiretroviral drugs (e.g., an antiretroviral therapy) and one or more statin compounds. In some embodiments, subjects diagnosed with HIV infection are administered a therapeutic combination of one or more antiretroviral drugs or a combination of antiretroviral drugs (e.g., an antiretroviral therapy) and one or more statin compounds prophylactically to prevent or delay the onset of HIV-associated PAH, or to reduce the severity of HIV-PAH and its symptoms. In some embodiments, subjects diagnosed with or having HIV-associated PAH are administered a therapeutic combination of one or more antiretroviral drugs or a combination of antiretroviral drugs (e.g., an antiretroviral therapy) and one or more statin compounds to treat PAH. In some embodiments, treating PAH encompasses slowing the onset or progression of the condition or ameliorating or reducing one or more symptoms associated with PAH. Combinations as described herein can also help people live more comfortably by preventing or ameliorating HIV-associated PAH, which can cause pain or discomfort, lead to debilitating effects, and cause morbidity. In some embodiments, a pharmaceutical composition as described herein comprises a combination of an antiretroviral therapy (or one or more antiretroviral agents) and one or more statin compounds and is administered to a subject prior to contracting HIV or following HIV infection.

Treatment will be suitably administered to subjects suffering from, having, susceptible to, having a propensity for, or at risk of developing, HIV-associated PAH. Determination of those subjects “at risk” can be made by any objective or subjective determination by a diagnostic test or opinion of a subject or health care provider (e.g., genetic test, enzyme or protein marker, and the like). Identifying a subject in need of such treatment can be in the judgment of a subject or a health care professional and can be subjective (e.g. opinion) or objective (e.g. measurable by a test or diagnostic method). Methods for administering combination therapies (e.g., concurrently or otherwise) are known to the skilled artisan and are described for example in Remington's Pharmaceutical Sciences by E. W. Martin.

### Statins—Inhibitors of HMG-CoA Reductase

Statins, e.g., 3-hydroxy-3-methylgluaryl coenzyme A (HMG-CoA) reductase inhibitors, can suppress inflammation and have markedly improved morbidity and mortality in clinical trials of disease and transplantation. In general, statins prevent the production of cholesterol in the liver by blocking HMG-CoA reductase. Statins reduce total cholesterol as well as low density lipoprotein (LDL) cholesterol in blood. LDL cholesterol (often referred to as “bad” cholesterol) is primarily responsible for the development of coronary artery disease. Reducing LDL cholesterol levels inhibits progression or even reverse coronary artery disease. In some cases, statins have ameliorated disease in experimental models of autoimmunity independently of their lipid lowering effects.

Treatment with rovastatin was reported to prevent pulmonary decline in patients with HIV-associated COPD. Without wishing to be bound by theory, statins, through their pleiotropic functions, may mitigate PAH pathogenesis by maintaining vascular cell homeostasis and preventing inflammatory feedback cascades that promote aberrant proliferation and vessel occlusion. The results from clinical trials of statins in the HIV-uninfected PAH population have been contradictory. In some cases, moderate improvement in PAH-associated biomarkers was reported, but long term physiologic benefits were not generally evident. The results obtained from studies of individuals who are not infected with HIV may not be generalized to HIV-associated PAH populations, in which chronic immune activation and inflammation are believed to play key roles in the development of cardiopulmonary co-morbidities.

Until recently, a lack of adequate animal models that faithfully recapitulate the immunologic, histologic, and hemodynamic features of human PAH has inhibited understanding of the disease pathobiology and has hindered the development of novel therapeutic strategies. Rodent models of PAH have provided key insights into PAH pathogenesis, but do not mimic the complex immunological dysregulation that contributes to idiopathic, autoimmune, and SIV- or HIV-PAH. Chimeric SHIV and SIV infected macaques have been reported to develop pulmonary arterial lesions, similar to patients with idiopathic PAH characterized by intimal and medial thickening with luminal occlusion.

In accordance with the present disclosure, the efficacy of statins on preventing or ameliorating SIV-PAH in rhesus macaques through longitudinal hemodynamic measurements and analysis of inflammatory signatures were evaluated. As described and exemplified herein, treatment of healthy macaques with a statin, e.g., atorvastatin, prior to and throughout SIV infection lowered the prevalence of SIV-PAH following SIV infection to 14.3% in treated macaques (2 of 14) compared to 52.2% among untreated controls (10 of 21). Moreover, statin treatment prevented SIV-PAH associated increases in the inflammatory cytokines TGF-β, MIP-1α, and TNF-α, and increased frequencies of CD14^{dim}CD16⁺ non-classical monocytes, and CCR7⁻CD163⁻CD206⁺ BALF macrophages previously shown to be associated with SIV-PAH. In addition, SIV-infected macaques treated with a combination of one or more statins and one or more antiretroviral agents in the post-acute phase were similarly protected from developing SIV-PAH (7.1%, 1 of 14). Although statins have known anti-inflammatory effects, treatment of infected animals did not significantly alter peripheral CD4⁺ T cells or viremic control. The compositions and methods described herein provide an optimal therapeutic balance whereby SIV-PAH is prevented by specifically curbing detrimental inflammatory pathways, without significantly altering viremic control.

As described herein, in some embodiments, a statin may be administered or combined with another drug or therapeutic (in addition to one or more antiretroviral agents or an antiretroviral therapy). By way of example, other therapeutics suitable for use include, without limitation, Caduet, which comprises atorvastatin formulated with amlodipine, a calcium channel blocker for high blood pressure; Advicor, which comprises lovastatin and niacin (nicotinic acid); Vytorin, which comprises simvastatin and ezetimibe, a cholesterol absorption inhibitor; and Simcor, which comprises simvastatin and niacin (nicotinic acid).

### Anti-PAH Prophylactics and Therapeutics

One or more statins, when administered in combination with an antiretroviral therapy (e.g., emtricitabine, tenofovir disoproxil fumarate), are useful as prophylactics and therapeutics for the prevention or treatment of HIV-associated PAH.

For therapeutic and prophylactic uses, the compositions comprising one or more statins as described herein and one or more antiretroviral agents as described herein may be administered systemically, for example, formulated in a pharmaceutically-acceptable buffer such as physiological saline. Nonlimiting routes of administration include, for example, subcutaneous (SC), intravenous (IV), intraperitoneal, intramuscular, or intradermal injections or modes of administration that provide continuous, sustained levels of the drug(s) in a patient. Treatment of human patients or other animals are carried out using a therapeutically effective amount of a combination therapeutic in a physiologically-acceptable carrier. Suitable carriers and their formulations are described, for example, in the most recent edition of Remington's Pharmaceutical Sciences by E. W. Martin. The amount of the therapeutic agent to be administered varies depending upon the manner and mode of administration, the age and body weight of the patient, and the clinical symptoms of viral infection and PAH. Generally, amounts will be in the range of those used for other agents used in the treatment of other diseases associated with HIV infection, although in certain instances, lower amounts may be needed because of properties of the composition and the therapeutic agents therein. Compositions and compounds (therapeutic agents) are administered at a dosage that controls the clinical or physiological symptoms of HIV-associated PAH, as may in some cases be determined by a diagnostic method known to one skilled in the art, or using any assay that measures the biological activity of a statin and/or an antiretroviral agent, such as emtricitabine, tenofovir disoproxil fumarate, or combination thereof.

In some embodiments, one or more statins are combined or co-formulated with one or more antiretroviral agents in a pharmaceutical composition and administered as such. In some embodiments, one or more statins are administered to a subject before, after, or concomitant with the administration of one or more antiretroviral agents or an antiretroviral therapy.

Therapeutic compounds and therapeutic combinations are administered in an effective amount. In certain embodiments, compounds such as those described herein, are administered at dosage levels of about 0.0001 to 4.0 g once per day (or multiple doses per day in divided doses) for adults. Thus, in certain embodiments, a compound or composition as described herein is administered at a dosage of any dosage range in which the low end of the range is any amount between 0.1 mg/day and 400 mg/day and the upper end of the range is any amount between 1 mg/day and 4000 mg/day (e.g., 5 mg/day and 100 mg/day, 150 mg/day and 500 mg/day). In other embodiments, a compound or composition as herein is administered at a dosage range in which the low end of the range is any amount between 0.1 mg/kg/day and 90 mg/kg/day and the upper end of the range is any amount between 1 mg/kg/day and 100 mg/kg/day (e.g., 0.5 mg/kg/day and 2 mg/kg/day, 5 mg/kg/day and 20 mg/kg/day). Preferably, a combination of the invention is administered at a dosage of 1.5 mg/kg/day, 15 mg/kg/day, 30 mg/kg/day. The dosing interval can be adjusted according to the needs of individual patients. For longer intervals of administration, extended release or depot formulations can be used. Table 3 provides exemplary amounts for administration of antiretroviral therapy (ART) (i.e., Emtricitabine, and Dolutegravir (DTG, Tivicay)). Table 4 provides exemplary amounts for administration of a statin (atorvostatin (Lipitor)).

TABLE 3

| | | | Average |
|---|---|---|---|
| ART Proposed | | | Weight of |
| cART Regimen for | | | monkeys (5.35 |
| Rhesus Macaques | Drug Type | Dosing mg/kg | kg) mg dosing |
| Emtricitabine | nucleoside | 50 mg/kg, | 268 mg |
| | analogue reverse | subcutane- | |
| | transcriptase | ously, | |
| | inhibitor (NRTI) | daily | |
| Tenofovir | nucleoside | 5.1 mg/kg, | 27 mg |
| disoproxil | analogue reverse | subcutane- | |
| fumarate | transcriptase | ously, | |
| | inhibitor (NRTI) | daily | |
| Dolutegravir | Integrase inhibitor | 2.5 mg/kg, | 13 mg |
| (DTG, | | subcutane- | |
| Tivicay) | | ously, | |
| | | daily | |

TABLE 4

| | | | AVG weight of |
|---|---|---|---|
| Statin | | | B6706 Monkeys |
| Treatment | | | (10.9 kg) mg/kg |
| for SIV-PAH | Drug Type | mg dosing | dosing |
| Atorvostatin | HMG-CoA | 10 mg animal | 0.9 mg/kg |
| (Lipitor) | Reductase Inhibitor | | |

### Formulation of Pharmaceutical Compositions

The administration of a compound for the treatment of HIV-associated PAH may be by any suitable means that results in a concentration of the therapeutic compound that, combined with another component or components, is effective in ameliorating, treating, or preventing HIV-associated PAH. Compounds comprising a composition may be contained in the appropriate amount in any suitable carrier substance, and are generally present in an amount of 1-95% by weight of the total weight of the composition. In an embodiment, the composition is provided in a dosage form that is suitable for oral administration. The pharmaceutical composition may be formulated according to conventional pharmaceutical practice (see, e.g., Remington: The Science and Practice of Pharmacy (20t^{h} ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions as described herein may be formulated to release the active compound substantially immediately upon administration or at any predetermined time or time period after administration. The latter types of compositions are generally known as controlled release formulations. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition, which, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Solid Dosage Forms for Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Tablets may be uncoated or they may be coated by known techniques, optionally, to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

Solid tablet compositions may include a coating adapted to protect the composition from unwanted chemical changes, (e.g., chemical degradation prior to the release of the active therapeutic substance). The coating may be applied on the solid dosage form in a similar manner as that described in Encyclopedia of Pharmaceutical Technology, supra.

At least two active therapeutics may be mixed together in a tablet, or may be partitioned. In one example, emtricitabine and tenofovir disoproxil fumarate comprise a single tablet. In some embodiments, the emtricitabine and tenofovir disoproxil fumarate are contained on the inside of the tablet, and the statin is on the outside, such that a substantial portion of the statin is released prior to the release of the emtricitabine and tenofovir disoproxil fumarate. Alternatively, the statin can be on the inside of the tablet, and the emtricitabine and tenofovir disoproxil fumarate are on the outside.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the active therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Diagnostic Methods

Patients who would likely benefit from the therapeutic methods as described herein are individuals who are identified as HIV-positive; or individuals who are at risk of being infected by HIV, or individuals having a propensity to develop HIV-associated PAH. Methods for identifying such patients are known to the skilled artisan. For example, individuals or patients having a propensity to develop HIV-associated PAH are those who engage in behavior that can increase the likelihood of HIV infection (e.g., needle sharing, unprotected sex, and the like); individuals or patients who have received an HIV-contaminated blood transfusion; or individuals or patients who were exposed to the virus in utero or through breast feeding. HIV infection can be diagnosed by detecting one or more HIV proteins or nucleic acids encoding one or more HIV proteins (e.g., the gag, pol, or env proteins) using methods and procedures commercially available and known to those having skill in the art.

### Combination Therapies

In some embodiments, compositions as described herein may be provided together with any other conventional anti-HIV or anti-retroviral therapy known in the art. For example, the compositions as described herein may be used or provided in conjunction with reverse transcriptase inhibitors, protease inhibitors, fusion inhibitors, or integrase inhibitors not co-formulated in the composition. Additionally, a person having or having a propensity to develop an HIV-associated disease such as PAH (e.g., one who is identified as having or at risk of developing an opportunistic infection or other malady associated with suppressed immune system function) may receive prophylactic treatment to inhibit or delay the development of the HIV-associated disease such as PAH that may be associated with the infection or malady.

### Patient Monitoring

The disease state, or success of treatment of a patient having HIV-associated PAH, can be monitored during treatment involving the administration of a composition as described herein. Such monitoring may be useful, for example, in assessing the efficacy of the treatment involving administration of a composition as described herein, or the efficacy of a particular statin, antiretroviral agent, or combination thereof, in treating a patient, such as an HIV-PAH patient. Therapeutics having anti-PAH activity as described herein are particularly advantageous for treating or preventing HIV-PAH and its symptoms and serious health effects as described.

### Kits

The invention provides kits for the treatment or prevention of HIV-associated PAH or symptoms thereof. In one embodiment, the kit includes a pharmaceutical pack comprising an effective amount of one or more antiretroviral agents and any one or more of the following statins: atorvastatin, fluvastatin, lovastatin, pravastatin, rosuvastatin, simvastatin, cerivastatin, mevastatin, pitavastatin, or a combination thereof. In some embodiments, the antiretroviral agent is Zidovudine (Retrovir, AZT), Didanosine (Videx, Videx EC, ddI), Stavudine (Zerit, d4T), Lamivudine (Epivir, 3TC), Abacavir (Ziagen, ABC), Tenofovir, a nucleotide analog (Viread, TDF), Lamivudine, Combivir (combination of zidovudine and lamivudine), Trizivir (combination of zidovudine, lamivudine and abacavir), Emtricitabine (Emtriva, FTC), Truvada (combination of emtricitabine and Tenofovir (tenofovir disoproxil fumarate), Epzicom (combination of abacavir and lamivudine), Nevirapine (Viramune, NVP), Delavirdine (Rescriptor, DLV), Efavirenz (Sustiva or Stocrin, EFV, also part of Atripla), Etravirine (Intelence, ETR), Rilpivirine (Edurant, RPV, also part of Complera or Epivlera), Saquinavir (Invirase, SQV), Indinavir (Crixivan, IDV), Ritonavir (Norvir, RTV), Nelfinavir (Viracept, NFV), Amprenavir (Agenerase, APV), Lopinavir/ritonavir (Kaletra or Aluvia, LPV/RTV), Atazanavir (Reyataz, ATZ), Fosamprenavir (Lexiva, Telzir, FPV), Tipranavir (Aptivus, TPV), Darunavir (Prezista, DRV), Enfuvirtide (Fuzeon, ENF, T-20), Maraviroc (Selzentry or Celsentri, MVC), Raltegravir (Isentress, RAL), Elvitegravir (EVG, part of the combination Stribild), Dolutegravir (Tivicay, DTG), emtricitabine, tenofovir, tenofovir disoproxil fumarate, or combination thereof. In some embodiments, the one or more antiretroviral agents and the one or more statins are present or co-formulated in a pharmaceutical composition. In some embodiments, the one or more antiretroviral agents and the one or more statins are present in separate pharmaceutical compositions that may be co-administered or administered at predetermined times. Preferably, the compositions are in unit dosage form. In some embodiments, the kit comprises a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired compositions or combinations thereof are provided together with instructions for administering them to a subject having or at risk of developing HIV-associated PAH. The instructions will generally include information about the use of the compounds for the treatment or prevention of HIV-associated PAH. In other embodiments, the instructions include at least one of the following: description of the compound or combination of compounds: dosage schedule and administration for treatment of HIV-associated PAH or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

The following examples are provided to illustrate the embodiments and aspects of the disclosure as described herein, and are not intended to be limiting. Those skilled in the art will understand that the specific constructions provided below may be changed in numerous ways, consistent with the description provided herein while retaining the critical properties of the compositions, or combinations thereof, and methods.

### EXAMPLES

### Example 1: Early Statin Treatment Prevents PAH in SIV-Infected NHPs

To determine the effect of statin treatment on PAH, SIV-infected rhesus macaques were studied. More specifically, a non-human primate (NHP) model of HIV-associated PAH that closely mimics HIV-PAH using simian immunodeficiency virus (SIV)-infected rhesus macaques (Macaca mulatta) was used. The SIV-PAH model displays an incomplete penetrance with 40-50% of infected macaques developing evidence of early stage PAH, thus mirroring the incomplete penetrance of PAH with HIV infection in humans. Moreover, the histologic and hemodynamic manifestations of SIV-PAH closely approximate those of human PAH. It was determined that treatment of healthy macaques with the statin atorvastatin prior to and throughout SIV infection prevented the development of SIV-associated PAH. Additionally, SIV-infected macaques that initiated atorvastatin treatment during the early chronic disease stage had reduced incidence of PAH compared to untreated animals. Statin treatment reduced inflammatory mediators, the pro-inflammatory monocytes and macrophages, and phenotypes associated with SIV-PAH. These results support the use of statins for the prevention of PAH in that statin treatment during early HIV-infection may reduce inflammatory processes that contribute to PAH and may provide a safe and effective therapeutic strategy to reduce the prevalence of HIV-PAH in HIV infected individuals.

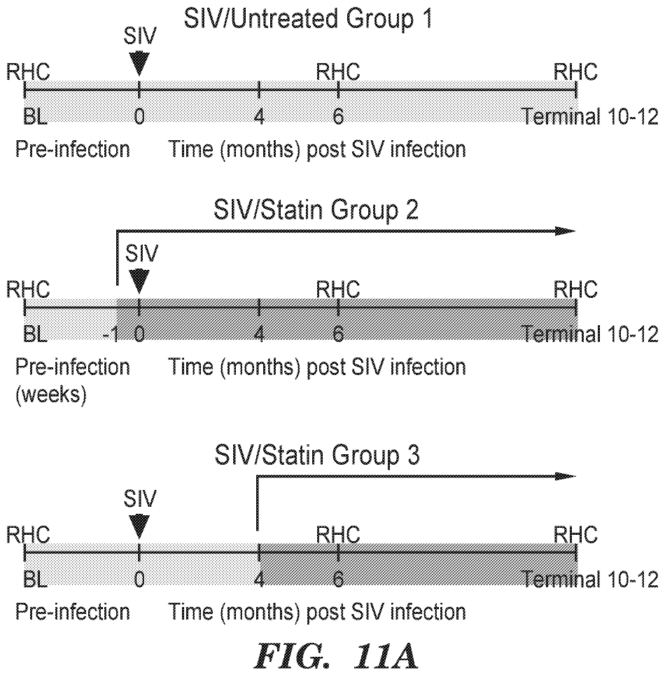

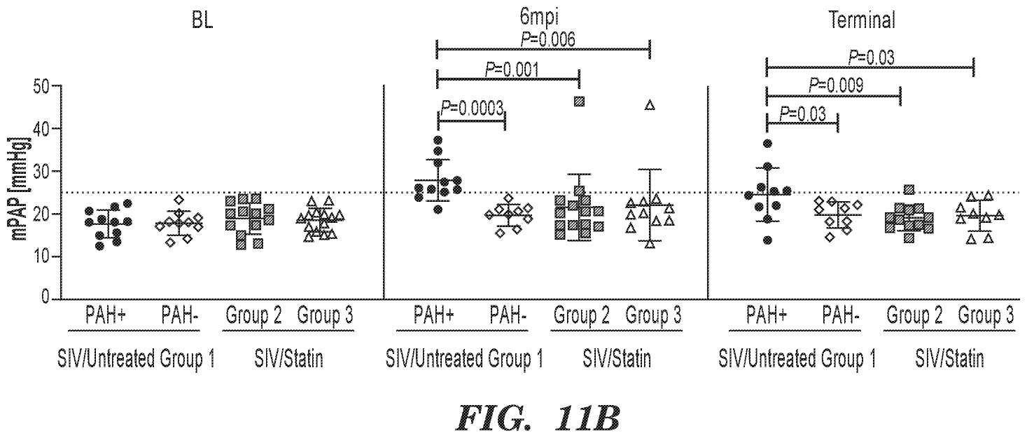

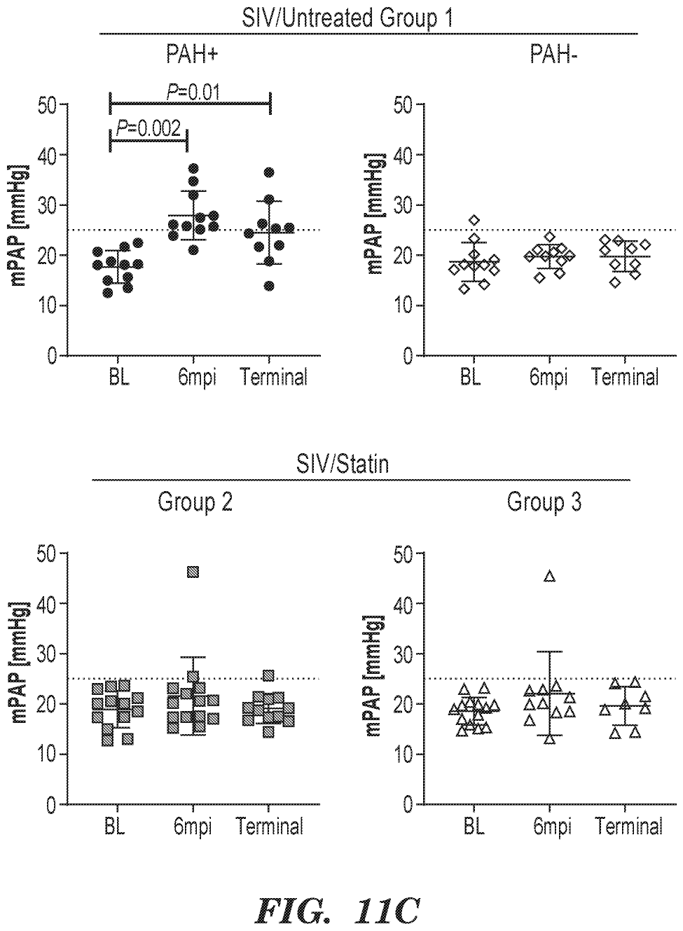

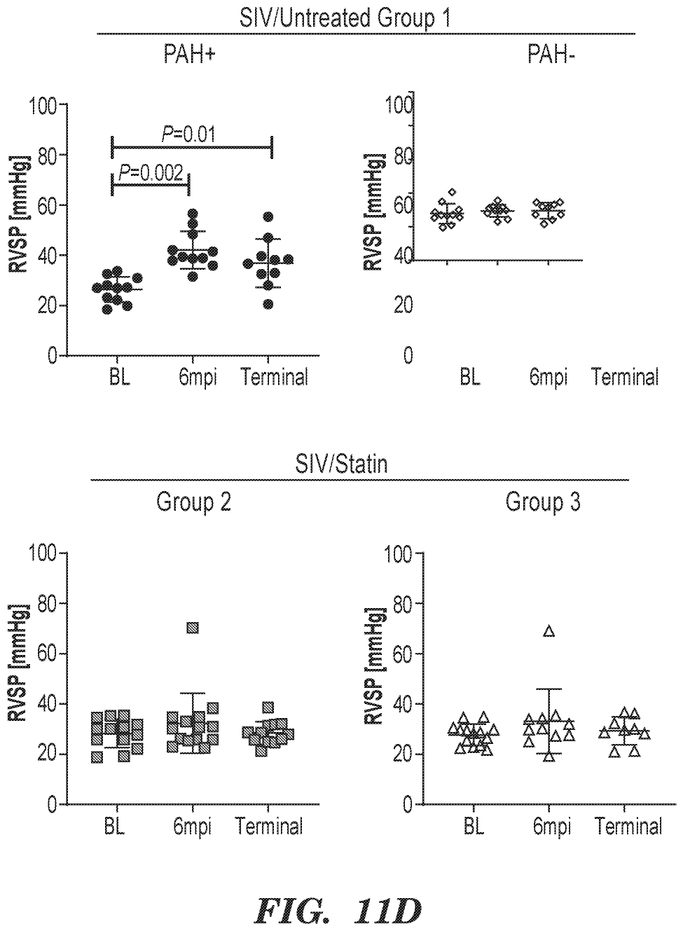

In this Example, three groups of macaques were assessed to determine the impact statins had on PAH onset and progression (FIGS. 11A, 11B). 52.2% (11 of 21) of SIV-infected macaques develop elevated pulmonary pressures within 6-12 months post infection (mpi) (Untreated/SIV Group 1 PAH⁺); whereas 47.6% (10 of 21) maintained normal hemodynamic parameters throughout the course of infection (up to 12 months post-infection) (Untreated/SIV Group 1 PAH) (Tables 5 and 6; FIGS. 11B-11D).

TABLE 5
Association between PAH and statin
treatment in SIV-infected macaques

| | SIV/ | | |
|---|---|---|---|
| | Untreated | SIV/Statin | SIV/Statin |
| Cohort | Group 1 | Group 2 | Group 3 |
| All animals | n = 21 | n = 14 | n = 14 |
| Primary Outcome | | | |
| Incidence PAH ≥ 25 mm Hg | 11 | 2 | 1 |
| 6-12 mpi | | | |
| Prevalence of PAH (% | 52.2 | 14.3 | 7.1 |
| PAH+) | | | |
| Relative Risk of PAH with | | | |
| statin treatment | | | |
| Value | — | 0.273 | 0.136 |
| 95% CI † | — | 0.073-0.859 | 0.024-0.653 |
| Association between PAH | | | |
| and statin treatment | | | |
| P value ‡ | — | 0.034* | 0.0097** |

CI, confidence interval;
mpi, months post infection;
PAH, pulmonary arterial hypertension
*P < 0.05,
**P < 0.01
† Koopman asymptotic score
‡ Fisher's exact test

TABLE 6
Clinical characteristics of SIV-infected rhesus macaques

| | | | | | | | | | |
|---|---|---|---|---|---|---|---|---|---|
| | | | Weight | Baseline | Chronic | Peak viral | Viral set | Peak | |
| | | Age at | at | blood | CD4+ | load (no. | point (no. | mPAP | |
| | | infection | infection | pressure | (no. of | of RNA | of RNA | (mm | PAH |
| Animal | Sex | (y) | (kg) | (mm Hg) | cells/μL) | copies/mL) | copies/mL) | Hg) | Status |

[symbol]

/Untreated Group 1

| | | | | | | | | | |
|---|---|---|---|---|---|---|---|---|---|
| 2813 | Female | 6.6 | 6.2 | 100/60 | 677 | 1.2 × 10⁷ | 1.8 × 10⁴ | 25.5 | PAH+ |
| 2913 | Female | 4.7 | 6 | 110/50 | 731 | 3.2 × 10⁶ | 2.8 × 10⁵ | 23.1 | PAH− |
| 3013 | Female | 6.1 | 7 | 110/60 | 330 | 2.0 × 10⁷ | 6.7 × 10⁴ | 15.5 | PAH− |
| 3113 | Female | 6.7 | 5 | 100/75 | 886 | 3.6 × 10⁸ | 1.2 × 10⁷ | 20.6 | PAH− |
| 3213 | Female | 5.3 | 6.4 | 110/70 | 297 | 2.4 × 10⁷ | 2.1 × 10⁶ | 27.9 | PAH+ |
| 3313 | Female | 6.1 | 8 | 110/70 | 701 | 4.4 × 10⁷ | 2.0 × 10⁶ | 26.1 | PAH+ |
| 3413 | Female | 4.9 | 5.4 | 120/65 | 440 | 3.9 × 10⁷ | 5.9 × 10⁵ | 34.7 | PAH+ |
| 3513 | Female | 4.7 | 7.4 | 110/60 | 274 | 1.1 × 10⁸ | 3.5 × 10⁵ | 23.7 | PAH− |
| 3613 | Female | 4.7 | 7 | 110/70 | 312 | 1.4 × 10⁷ | 2.7 × 10⁶ | 21.4 | PAH− |
| 6513 | Male | 5.8 | 11.8 | 110/60 | 690 | 1.7 × 10⁷ | 1.9 × 10⁵ | 21.4 | PAH− |
| 10413 | Male | 5.8 | 8.8 | 110/75 | 397 | 1.2 × 10⁸ | 1.2 × 10⁷ | 21.0 | PAH− |
| 10613 | Male | 5.7 | 7.2 | 130/80 | 449 | 3.8 × 10⁶ | 3.3 × 10⁶ | 31.1 | PAH+ |
| 10713 | Male | 6.9 | 6.8 | 135/95 | 378 | 1.2 × 10⁶ | 1.1 × 10⁶ | 21.1 | PAH− |
| 11313 | Male | 5.8 | 8 | 140/80 | 679 | 1.0 × 10⁷ | 2.2 × 10² | 23.0 | PAH− |
| 11413 | Male | 5.8 | 8.8 | 130/70 | 188 | 7.1 × 10⁶ | 7.9 × 10⁵ | 18.9 | PAH− |
| 11613 | Male | 5.9 | 7.4 | 130/80 | 447 | 1.2 × 10⁸ | 2.2 × 10⁶ | 26.3 | PAH+ |
| 17511 | Male | 7.3 | 11.8 | 140/80 | 413 | 4.8 × 10⁶ | 3.5 × 10⁴ | 37.3 | PAH+ |
| 17611 | Male | 7.1 | 10.8 | 120/70 | 128 | 1.1 × 10⁷ | 1.0 × 10⁶ | 25.2 | PAH+ |
| 18011 | Male | 7.9 | 10.2 | 110/50 | 733 | 2.6 × 10⁶ | 2.1 × 10⁵ | 36.5 | PAH+ |
| 18211 | Male | 8.3 | 12.4 | 130/90 | 372 | 1.0 × 10⁷ | 1.3 × 10⁶ | 32.0 | PAH+ |
| 18311 | Male | 7.2 | 11.6 | 110/50 | 443 | 7.3 × 10⁶ | 9.5 × 10⁵ | 27.5 | PAH+ |
| [symbol] ean ± | | 6.1 ± | 8.3 ± | 118 ± 13/ | 474 ± | 4.5 ± 8.2 × | 2.0 ± 3.4 × | 25.7 ± | 52.2% |
| [symbol] or PAH | | 1.0 | 2.3 | 70 ± 12 | 205 | 10⁷ | 10⁶ | 5.8 | |
| [symbol] valence | | | | | | | | | |

[symbol]

/Statin Group 2

| | | | | | | | | | |
|---|---|---|---|---|---|---|---|---|---|
| 22-16 | Female | 9.0 | 6 | 120/90 | 833 | 8.1 × 10⁶ | 3.0 × 10⁴ | 15.2 | PAH− |
| 24-16 | Female | 11.0 | 5.3 | 110/80 | 416 | 7.9 × 10⁶ | 6.1 × 10³ | 46.3 | PAH+ |
| 30-16 | Male | 5.0 | 11.1 | 115/80 | 347 | 1.6 × 10⁷ | 1.8 × 10⁶ | 23.2 | PAH− |
| 33-16 | Male | 5.0 | 17 | 115/70 | 429 | 1.3 × 10⁷ | 1.7 × 10⁶ | 25.7 | PAH+ |
| 34-16 | Male | 9.0 | 15.3 | 100/70 | 231 | 7.3 × 10⁶ | 9.9 × 10⁵ | 19.2 | PAH− |
| 37-16 | Female | 6.0 | 7.8 | 100/60 | 1451 | 1.7 × 10⁷ | 2.7 × 10⁵ | 23.2 | PAH− |
| 38-16 | Female | 9.0 | 7.1 | 110/90 | 394 | 1.8 × 10⁷ | 1.3 × 10⁴ | 17.6 | PAH− |
| 39-16 | Female | 10.0 | 8.9 | 120/90 | 272 | 4.9 × 10⁷ | 1.7 × 10⁶ | 20.2 | PAH− |
| 41-16 | Female | 9.0 | 9.3 | 120/90 | 412 | 1.1 × 10⁷ | 8.8 × 10⁴ | 17.4 | PAH− |
| 46-16 | Male | 8.0 | 13.4 | 140/70 | 188 | 9.3 × 10⁶ | 3.5 × 10⁵ | 16.6 | PAH− |
| 47-16 | Male | 9.0 | 13.1 | 170/100 | 188 | 3.7 × 10⁷ | 4.6 × 10⁴ | 17.1 | PAH− |
| 48-16 | Male | 9.0 | 11.3 | 100/50 | 499 | 5.0 × 10⁷ | 2.9 × 10⁶ | 22.0 | PAH− |
| 50-16 | Male | 9.0 | 13.9 | 130/80 | 160 | 1.0 × 10⁷ | 1.4 × 10⁵ | 20.6 | PAH− |
| 52-16 | Male | 6.0 | 13.5 | 110/80 | 411 | 5.0 × 10⁶ | 1.2 × 10⁵ | 20.7 | PAH− |
| Mean ± | | 8.1 ± | 10.9 ± | 119 ± 19/ | 445 ± | 1.9 + 1.5 × | 7.3 ± 9.4 × | 21.8 ± | 14.3% |
| SD or PAH | | 1.9 | 3.6 | 79 ± 14 | 336 | 10⁷ | 10⁵ | 7.6 | |
| incidence | | | | | | | | | |

SIV/Statin Group 3

| | | | | | | | | | |
|---|---|---|---|---|---|---|---|---|---|
| 25-16 | Female | 10.0 | 6.2 | 125/100 | 261 | 2.4 × 10⁷ | 1.4 × 10⁵ | 21.4 | PAH− |
| 26-16 | Female | 9.0 | 6.9 | 130/90 | 851 | 1.6 × 10⁷ | 4.1 × 10³ | 16.8 | PAH− |
| 27-16 | Female | 9.0 | 6 | 115/60 | 517 | 1.2 × 10⁷ | 1.1 × 10⁶ | 13.2 | PAH− |
| 28-16 | Female | 9.0 | 7 | 120/90 | 986 | 4.7 × 10⁷ | 7.8 × 10² | 23.6 | PAH− |
| 29-16 | Male | 6.0 | 16 | 125/90 | 203 | 4.3 × 10⁷ | 1.4 × 10⁶ | 45.5 | PAH+ |
| 31-16 | Male | 9.0 | 9.5 | 90/65 | 222 | 2.2 × 10⁷ | 1.2 × 10⁷ | N/A | PAH− |
| 32-16 | Male | 9.0 | 10.1 | 120/95 | 713 | 1.0 × 10⁶ | 1.5 × 10⁴ | 22.9 | PAH− |
| 35-16 | Male | 10.0 | 9 | 120/95 | 307 | 8.4 × 10⁵ | 3.0 × 10² | 24.1 | PAH− |
| 36-16 | Male | 7.0 | 14.3 | 90/60 | 627 | 8.3 × 10⁶ | 1.1 × 10⁵ | 18.9 | PAH− |
| 43-16 | Female | 9.0 | 8.3 | 115/80 | 949 | 7.3 × 10⁶ | 1.8 × 10⁶ | 20.2 | PAH− |
| 44-16 | Female | 8.0 | 5.7 | 140/100 | 180 | 9.6 × 10⁶ | 4.4 × 10⁵ | 19.8 | PAH− |
| 45-16 | Male | 10.0 | 10.9 | 110/80 | 349 | 8.8 × 10⁶ | 2.8 × 10³ | 24.4 | PAH− |
| 49-16 | Male | 6.0 | 13 | 130/85 | 277 | 2.9 × 10⁷ | 4.0 × 10⁵ | N/A | PAH− |
| 51-16 | Male | 9.0 | 11.1 | 100/70 | 690 | 5.8 × 10⁶ | 9.0 × 10³ | 19.9 | PAH− |
| [symbol] ean ± | | 8.6 ± | 9.6 ± | 116 ± 15/ | 509 ± | 1.7 ± 1.4 × | 1.2 ± 3.1 × | 22.5 ± | 7.1% |
| SD or PAH | | 1.3 | 3.2 | 83 ± 14 | 290 | 10⁷ | 10⁶ | 7.9 | |
| incidence | | | | | | | | | |

mPAP, mean pulmonary arterial pressure;
N/A, mot available;
PAH, pulmonary arterial hypertension [symbol]
indicates data missing or illegible when filed

To determine if statin therapy in healthy macaques could alter the incidence or progression of SIV-PAH, atorvastatin treatment was initiated in a subset of non-human primates (NHPs) 1 week prior to SIV infection (FIG. 11A, SIV/Statin Group 2; Table 6). Hemodynamics were measured by serial right heart catheterizations at baseline (BL), 6 months post-infection (6 mpi), and at the end of study (10-12 mpi) (FIG. 11C, 11D). In contrast to untreated controls (Untreated/SIV Group 1), 14.3% (Table 5, 2 of 14, P=0.03) of statin-treated animals (SIV/Statin Group 2) developed elevated mean pulmonary arterial pressure (mPAP) (monkey 3316 at 25.4 mm Hg and monkey 2416 at 46.3 mm Hg) at 6 mpi (FIG. 1). Of these animals, one maintained slightly elevated mPAP until the end of the study (monkey 3316, terminal mPAP=25.7). At study termination, mPAP of monkey 2416 decreased to 21.2 mm Hg.

It was further tested whether statin treatment initiated following SIV infection could modify PAH incidence or progression (FIG. 11A, SIV/Statin Group 3; Table 6). In this cohort, 1 of 14 monkeys (Table 2, 7.1%, P=0.0097) developed PAH (animal 2916; 6 mpi mPAP=45.5) at 6 mpi. This monkey had a relatively high baseline mPAP (23.0 mm Hg) and was euthanized at 36 weeks post-infection (wpi) due to Pneumocystis pneumonia without terminal right heart catheterization (RHC). These results demonstrate that SIV-PAH can be prevented through prophylactic and therapeutic intervention with atorvastatin.

### Example 2: Effect of Statins on Peripheral CD4⁺ T Cells and SIV Infection

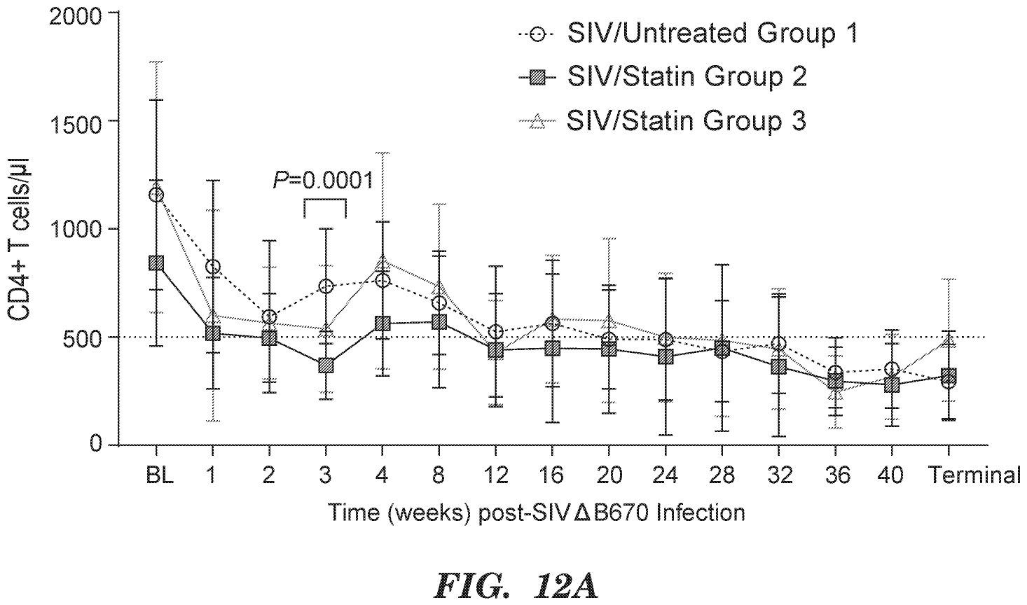

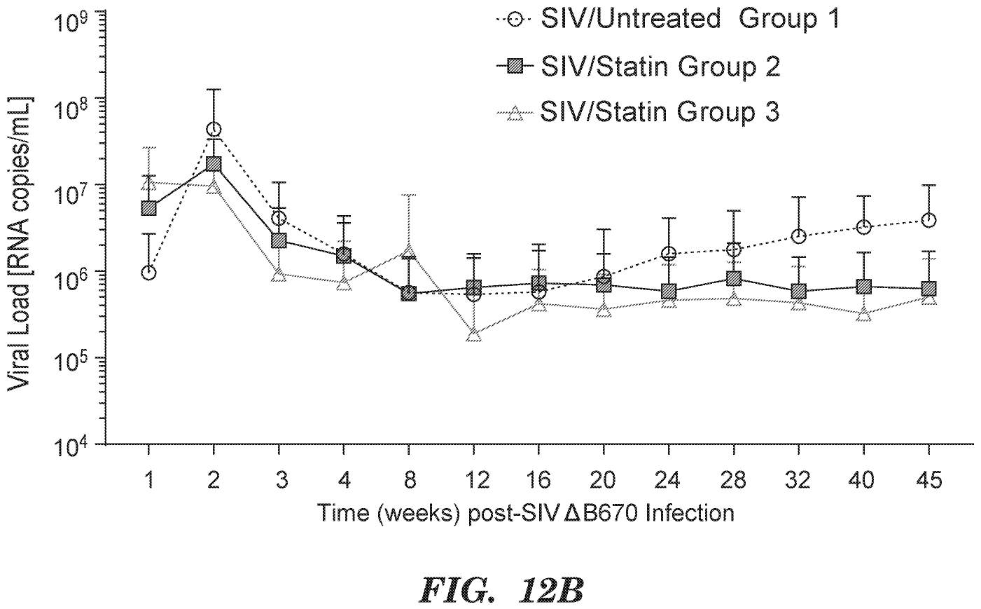

Statins have been reported to inhibit lymphocyte migration which in turn can impair T effector responses necessary for pathogen clearance and viremic control. Therefore, peripheral blood CD4⁺ T cell levels and plasma viral load throughout SIV infection were examined. CD4⁺ T cell frequency in both statin-treated cohorts were similar to SIV/Untreated controls except in SIV/Statin Group 1 at 3 weeks post infection (FIG. 12A, P=0.0001 compared to SIV/Untreated Group 1); however, cell numbers recovered by 8 wpi remained similar throughout the remainder of the experiment. In addition, statin treatment did not significantly alter viral load compared to SIV/Untreated controls (FIG. 12B). All SIV-infected macaques displayed the typical decline in peripheral blood CD4⁺ T cells and a characteristic chronic-phase plasma viral level. Together, these data indicate that statin treatment did not significantly alter peripheral CD4⁺ T cell homeostasis or viral load in this study.

### Example 3: Statin Treatment Prevents Alterations in Cytokine Profiles Associated with SIV-PAH

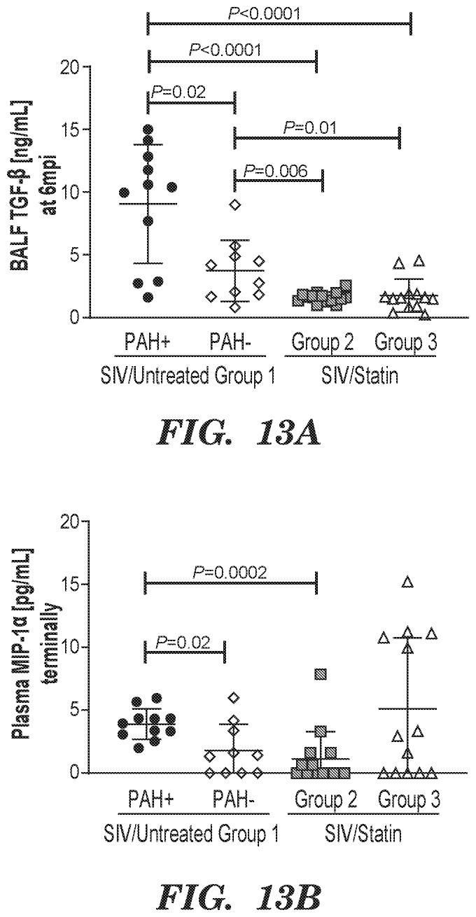

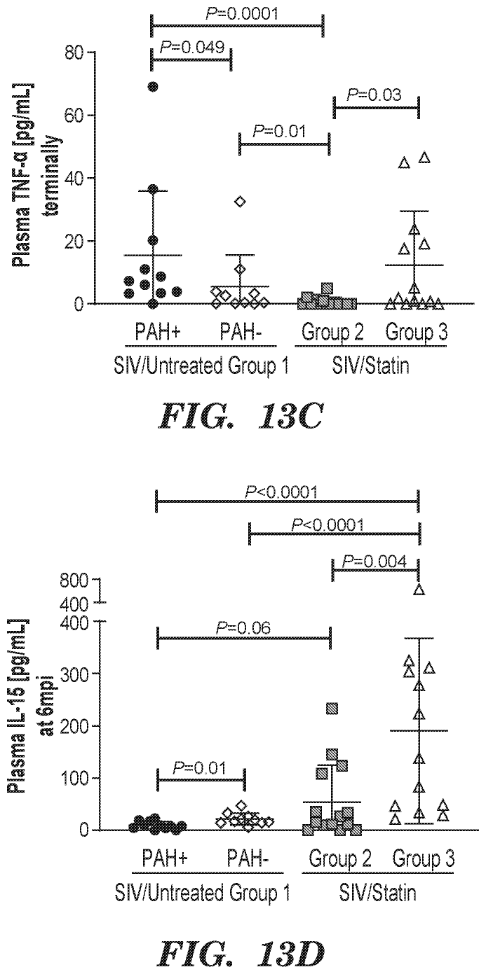

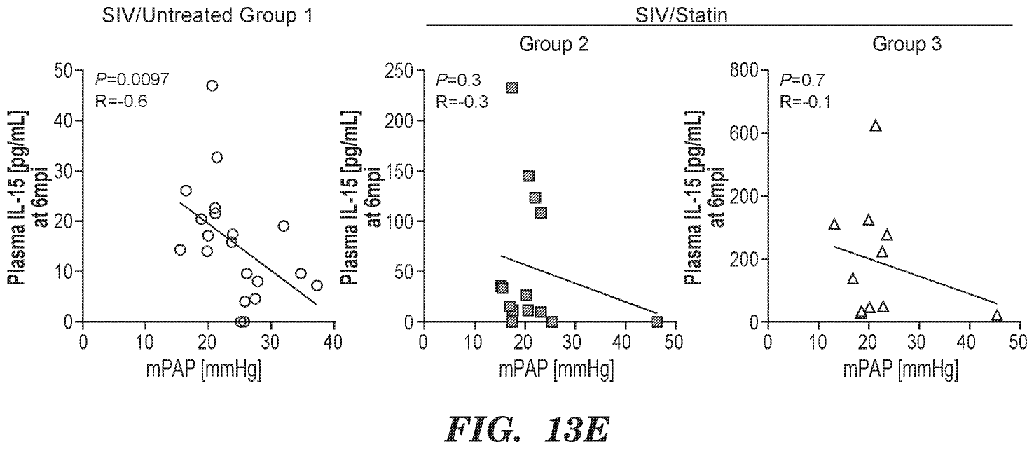

HIV induces a state of chronic inflammation that may drive PAH pathogenesis. Among inflammatory cytokines associated with HIV-PAH, BALF TGF-β (FIG. 13A, P=0.02) and plasma MIP-1α (FIG. 13B, P=0.02) and TNF-α (FIG. 13C, P=0.049) levels were significantly higher in SIV-PAH⁺ animals compared with SIV-PAH− controls. To determine if statin treatment could modify these inflammatory mediators associated with SIV-PAH, cytokine profiles in the statin-treated cohorts were compared. Consistent with the hypothesis that statin treatment can suppress SIV-PAH associated inflammation, levels of BALF TGF-β at 6 mpi were significantly lower in both statin-treated cohorts compared to PAH⁺ (FIG. 13A; SIV/Statin Group 2, P<0.0001; SIV/Statin Group 2, P<0.0001) and PAH⁻ SIV/Untreated controls (FIG. 13A; SIV/Statin Group 2, P=0.006; SIV/Statin Group 3, P=0.01). In addition, terminal levels of plasma MIP-1α (FIG. 13B, P=0.0002) and TNF-α (FIG. 13C, P=0.0001) were significantly lower in SIV/Statin Group 2 compared with SIV-PAH⁺ controls. However, terminal plasma levels of MIP-1α and TNF-α were not significantly reduced in SIV/Statin Group 3 indicating that these responses may not be suppressed when statin treatment is initiated during the post-acute phase of infection.

In contrast with inflammatory cytokines TGF-β, MIP-1α, and TNF-α, IL-15 was distinctly elevated in animals resistant to SIV-PAH (FIG. 13D, P=0.01) and inversely correlated with increased pulmonary pressures at 6 mpi (FIG. 13E; left panel, P=0.0097). Statin treatment increased IL-15 levels in both statin cohorts compared with SIV-PAH⁺ controls (FIG. 13D; SIV/Statin Group 2, P=0.06; SIV/Statin Group 3, P<0.0001). IL-15 levels in both statin cohorts did not correlate with increased pulmonary pressures (FIG. 13E; center, right panels). Collectively, these data indicate that statin treatment suppresses chronic inflammation and may promote cytokine responses associated with SIV-PAH resistance.

IL-15 is primarily associated with maintaining lymphocyte homeostasis, and chronic inflammation. In examining IL-15 levels in statin-treated cohorts as described in this Example, it was observed that statin treatment significantly increased levels of plasma IL-15 compared to levels in untreated controls and correlated with a decreased incidence of SIV-PAH. Interestingly, the three statin-treated animals that developed PAH had the lowest levels of IL-15 within their respective treatment cohorts; accordingly, lower levels of plasma IL-15 may increase risk for HIV-PAH. Methods of determining increased risk of HIV-PAH in subjects by detecting or measuring lower levels of IL-15 in a sample, such as a blood, plasma, or serum sample, compared with the levels in subjects having normal IL-15 levels may be beneficial for stratifying individuals having HIV as to their propensity or risk of developing PAH. The data indicate that upregulation of IL-15 is associated with protection from PAH.

### Example 4: Statin Treatment Prevents Monocyte and Macrophage Skewing Associated with Inflammation and Fibrosis in SIV-PAH

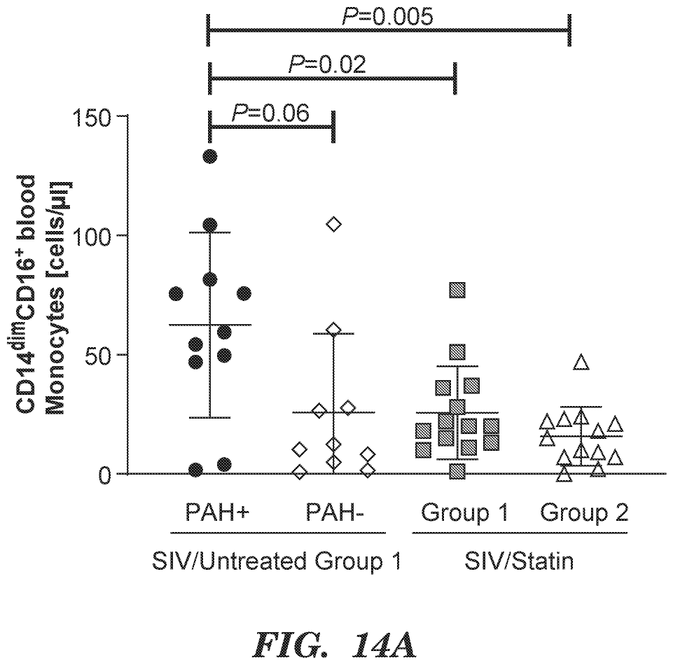

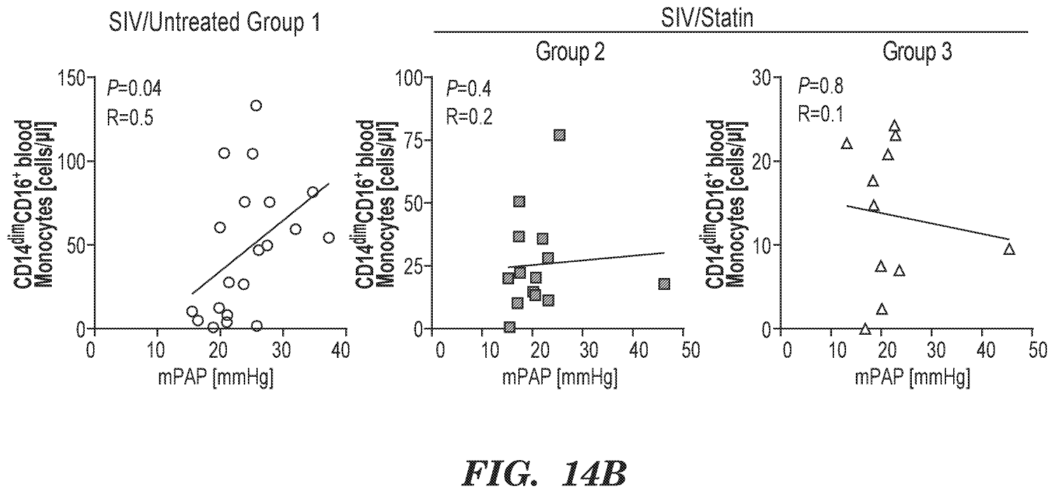

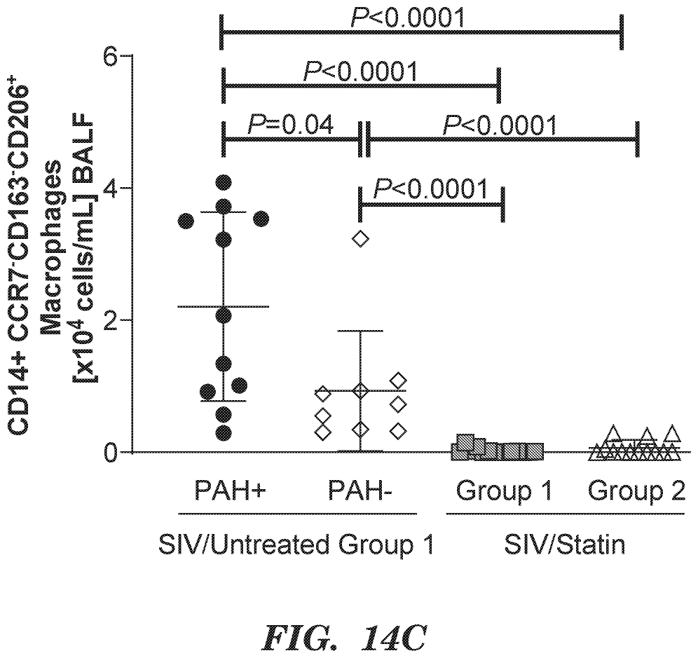

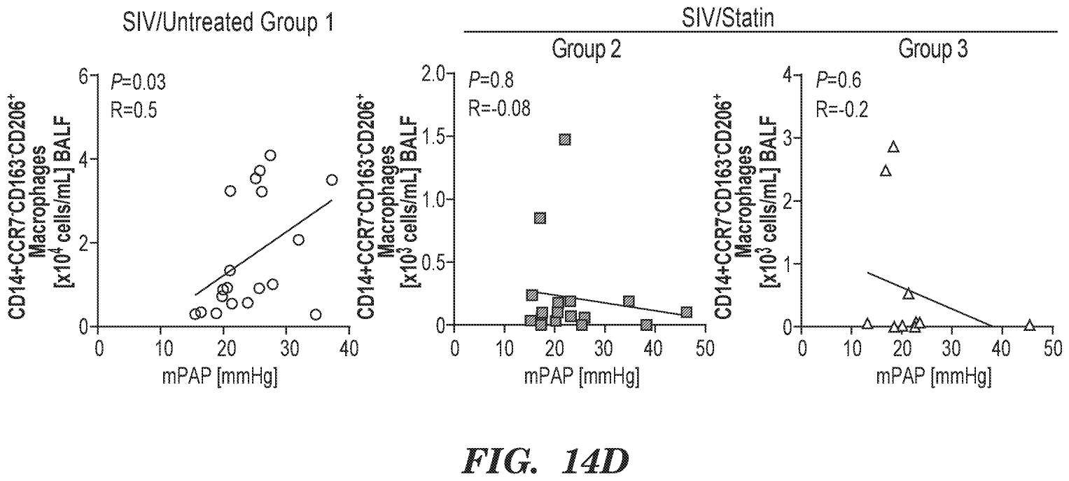

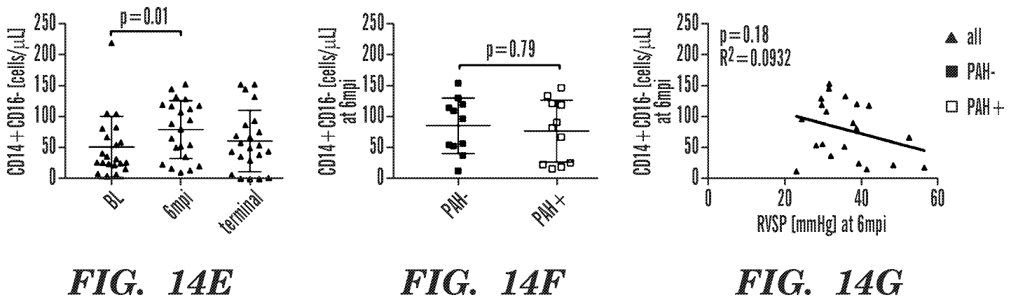

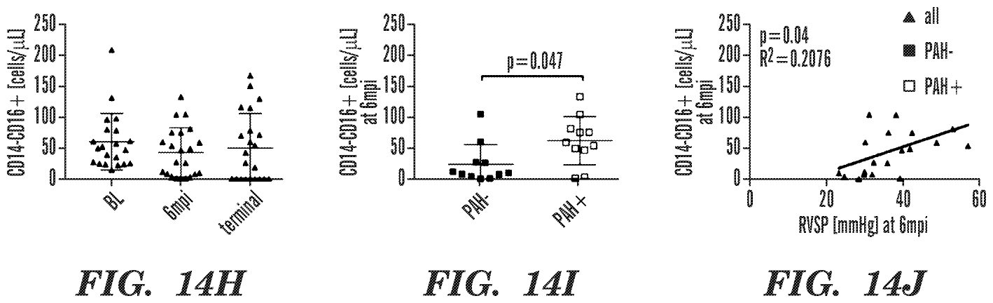

Among cytokine signatures associated with the SIV-PAH, MIP-1α, TNF-α, and TGF-β have been previously associated with macrophage populations that promote fibrosis. At 6 mpi, SIV-PAH⁺ animals had higher numbers of peripheral blood CD14^{dim}CD16⁺ non-classical monocytes (FIG. 14A, p=0.06) and CCR7⁻CD163⁻CD206⁺ BALF macrophages (FIG. 14C, p=0.04) compared to SIV-PAH⁻ controls. Moreover, increased numbers of CD14^{dim}CD16⁺ non-classical monocytes (FIG. 14B, left panel, p=0.04) and CD14⁺ CCR7⁻CD163⁻CD206⁺ macrophages (FIG. 14D, left panel, p=0.03) correlate with increased pulmonary pressures in SIV/Uninfected controls at 6 mpi. Given the pleiotropic effects of statin upon monocyte and macrophage skewing and cytokine secretion, it was hypothesized that statins may decrease these populations which are associated with SIV-PAH. Consistent with this hypothesis, the number of CD14^{dim}CD16⁺ non-classical monocytes was significantly lower in both statin cohorts compared to SIV-PAH⁺ controls (FIG. 14A; SIV/Statin Group 2, p=0.02; SIV/Statin Group 3, p=0.005) and did not correlate with increased pulmonary pressures (FIG. 14B, center, right panels). Moreover, the number of BALF CCR7⁻CD163⁻CD206⁺ BALF macrophages also were significantly reduced with statin treatment (FIG. 14C; SIV/Statin Group 2, p<0.0001; SIV/Statin Group 3, p<0.0001) compared to both PAH⁺ and PAH⁻ SIV/Untreated controls. Furthermore, these macrophage numbers did not correlate with increased pulmonary pressures (FIG. 14D, center, right panels). These data suggest that statin treatment reduces monocyte and macrophage phenotypic skewing associated with SIV-PAH.

Additionally, at 6 mpi, there were significantly more CD14⁺CD16⁻ cells than at baseline (FIG. 14E, p=0.01). However, no significant differences were observed for this cell type between PAH⁻ and PAH⁺ macaques (FIG. 14F, p=0.79). Additionally, a strong correlation between RSVP at 6 mpi and the number of CD14⁺CD16⁻ cells was not observed (FIG. 14G, p=0.18). While there were no observed statistically significant differences in the number of CD14⁺CD16⁻ cells at different timepoints of infection (FIG. 14H), greater numbers of these cells were observed in PAH⁺ cells compared to PAH⁻ cells (FIG. 14I, p=0.47). A correlation between RSVP at 6 mpi and the number of CD14⁺CD16⁻ cells was observed (FIG. 14J, p=0.04).

### Example 5: Statin-Treatment Prevents SIV-PAH-Associated Fibrosis in the Heart and Pulmonary Arteries

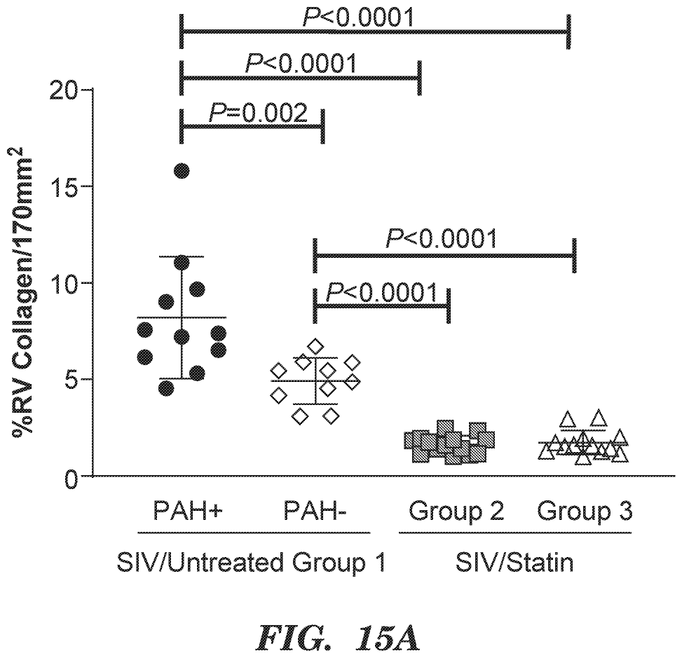

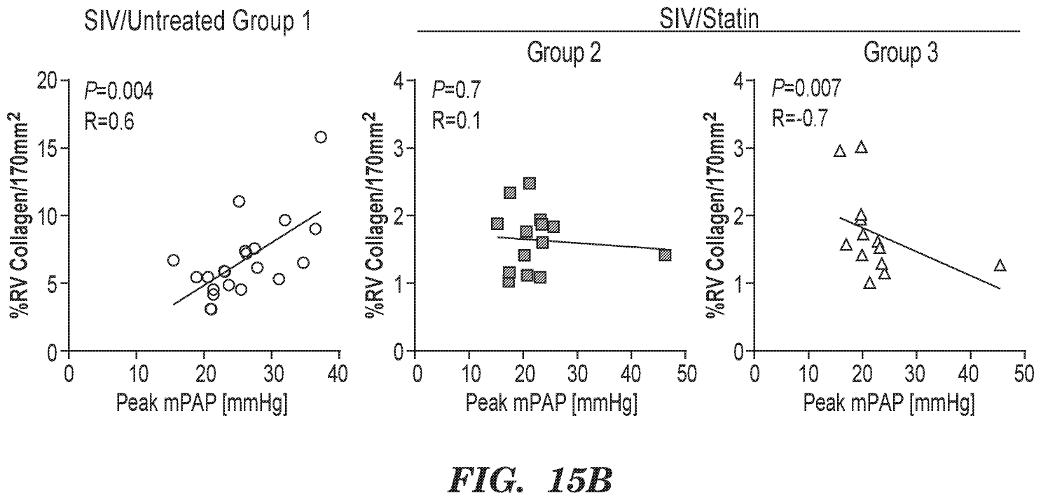

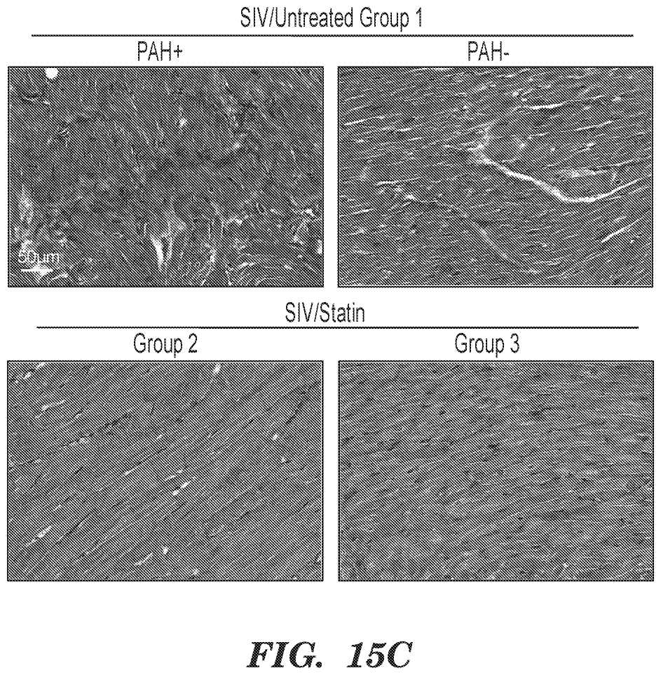

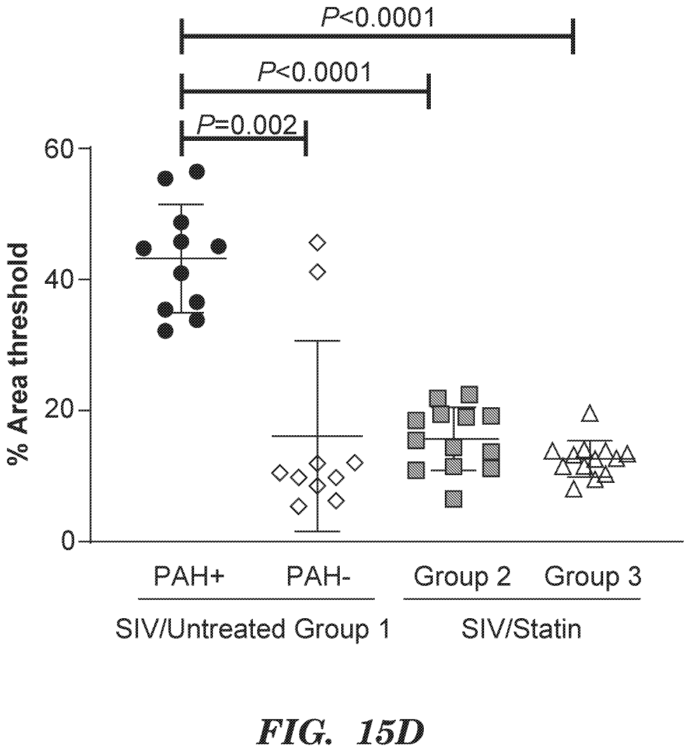

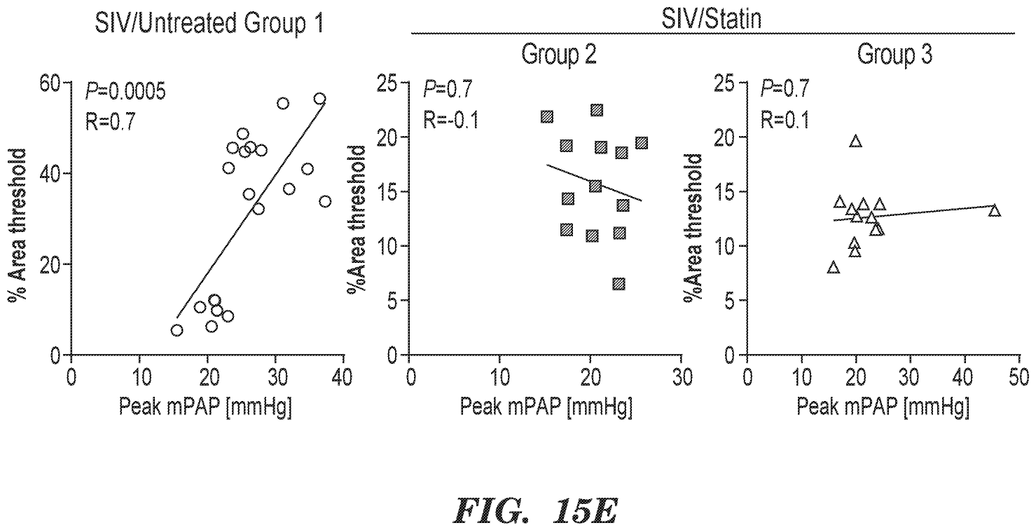

SIV-infected macaques exhibit pulmonary arterial lesions similar to idiopathic human PAH; however, the extent of vascular remodeling is relatively mild compared to HIV-PAH. Studies have indicated that right ventricular fibrosis is a relatively early manifestation of SIV-PAH and occurs in advance of significant pulmonary vascular lesions (Tarantelli, R. A. et al. Longitudinal Evaluation of Pulmonary Arterial Hypertension in a Rhesus Macaque (Macaca mulatta) Model of HIV Infection. Comparative medicine (2018)). SIV-PAH⁺ untreated controls had significantly higher levels of fibrosis within the right ventricle (FIG. 15A, P=0.002) and pulmonary arteries (FIG. 15d, P=0.002) compared with SIV-PAH− monkeys. Moreover, increases in collagen deposition within the right ventricle (FIG. 15B, P=0.004; FIG. 15C) and small pulmonary arteries (FIG. 15D, P=0.0005) correlated with increased pulmonary pressures in SIV/Uninfected controls. Given the substantial dampening of pro-fibrotic immune phenotypes that occurs with statin treatment, right ventricular and lung periarteriolar collagen deposition were examined following statin treatment. Within the right ventricle, both statin-treated cohorts had significantly less collagen deposition compared to SIV-PAH⁺ (FIG. 15D; SIV/Statin Group 2, P<0.0001; SIV/Statin Group 3, P<0.0001). Collagen deposition in both statin-treated cohorts did not correlate with peak pulmonary pressures as previously observed in SIV/Untreated controls (FIG. 15E, center and right panels). Similarly, the small pulmonary arteries of statin-treated cohorts had significantly less collagen deposition compared to SIV-PAH⁺ (FIG. 15D; SIV/Statin Group 2, P<0.0001; SIV/Statin Group 3, P<0.0001). Surprisingly, statin-treated cohorts also had considerably less collagen deposition in both the right ventricle and lung compared with SIV-PAH⁻ controls. These data reveal that statin intervention therapy significantly reduces fibrosis in the heart and pulmonary arteries during SIV-infection.

In this Example, collagen deposition in the right heart and in the pulmonary vasculature was examined to determine if statin treatment reduced fibrosis, as SIV-PAH is associated with increased right heart and lung periarteriolar fibrosis. The results showed that statin treatment prevented collagen deposition associated with SIV-PAH in both the right heart and pulmonary vasculature. Moreover, statin-treated cohorts had even less RV collagen deposition than SIV-PAH− animals. These data support the notion that statins prevent elevated pulmonary pressures during SIV- or HIV-infection by preventing fibrosis and can also prevent collagen deposition associated with SIV- or HIV-infection

The results of the Examples described herein demonstrate that SIV-PAH, as well as HIV-PAH, are preventable diseases that can be abrogated through pharmaceutical intervention, for example, up to the early chronic phase of infection and later. In addition, SIV-PAH pathogenesis was demonstrated to be driven by key immunologic processes that include specific inflammatory pathways and pro-fibrotic myeloid populations. Moreover, these processes can be curtailed through preventive therapy using statins, which are commercially available compounds, and which may be applicable to both HIV-infected and non-infected individuals (human patients) who are at risk of developing PAH.

While the full breadth of pathogenic mechanisms driving HIV-PAH is not fully known, statin treatment was shown as described supra to successfully inhibit several of the known PAH-associated immunologic parameters. The results obtained from the above Examples advantageously demonstrate the efficacy of statin prevention therapy in a highly relevant, pre-clinical, NHP model of HIV-PAH. In addition, the efficacy of statin therapy is demonstrated in the absence of confounding factors such as illicit drug use, previous antiretroviral therapy, and non-Pneumocystis co-infection. The data presented in the described Examples are clinically significant because they suggest that HIV-PAH can be prevented early in HIV infection by administering one or more statin drugs, as well as one or more antiretroviral agents (or ART), that are FDA approved, for the prevention and treatment of HIV-associated PAH.

### Example 6: Materials and Methods

Animals. 28 adult Chinese rhesus macaques (Macaca mulatta) aged 6-10 years old were obtained from national primate centers or vendors and housed in accordance with the NIH Guide for the Care and Use of Laboratory Animals in a BSL2⁺ primate facility at the University of Georgia (National Research Council Committee for the Update of the Guide for the Care and Use of Laboratory Animals, Guide for the Care and Use of Laboratory Animals (8^{th} ed.) (National Academies Press National Academy of Sciences., 2011). Prior to admission to the study, all animals were screened and found negative for simian retroviruses and preexisting cardiovascular disease.
Study Design and Statin treatment. All cohorts were infected with SIVΔB670 (1:100 in PBS), tissue culture infectious dose of 50% (TCID₅₀)=2.6×10⁵), intravenously or mucosally as previously described (Tarantelli et al. (2018); Schweitzer, F. et al. (2019)). To test the hypothesis that prophylactic or therapeutic treatment with atorvastatin could alter the incidence or progression of SIV-PAH, treatment was initiated in a subset of NHP (n=14) 1 week prior to SIV infection and in a second cohort (n=14) 4 months following SIV-infection. Statin-treated cohorts received 10 mg/day atorvastatin orally. All procedures were approved by the University of Georgia Institutional Animal Care and Use Committee.
Hemodynamic measurements through right hearth catheterization. Right heart catheterization was performed and analyzed using a Swan-Ganz balloon wedge pressure catheter advanced through the right atrium, right ventricle, and pulmonary artery as previously described (Tarantelli et al).
Flow Cytometry. Peripheral blood and bronchoalveolar lavage fluid (BALF) were collected at baseline (BL), 6 months post infection (mpi) and at study termination (10-12 mpi), and processed for flow cytometry as previously described (Board, K. F. et al. Experimental Pneumocystis carinii pneumonia in simian immunodeficiency virus-infected rhesus macaques. The Journal of infectious diseases 187, 576-588 (2003) and Kling, H. M. et al. Relationship of Pneumocystis jiroveci humoral immunity to prevention of colonization and chronic obstructive pulmonary disease in a primate model of HIV infection. Infection and immunity 78, 4320-4330 (2010)). CD14^{dim}CD16⁺ non-classical monocytes and CCR7⁻CD163⁻CD206⁺ BALF macrophages were identified by flow cytometry and calculated as previously described by Schweitzer, F. et al., supra). All analyses were performed using FlowJo Analysis software (Tree Star, Inc., Ashland, Oreg.).
Cytokine measurement in plasma and BALF. Quantitative analysis of cytokines and chemokines in the plasma and BALF was performed using the Cytokine 29-Plex Monkey Panel (Invitrogen, Carlsbad, Calif.) according to the manufacturer's instructions. BALF analytes were normalized on the assumption that plasma and BALF have equal urea concentrations as previously described, using Quanti Chrom Urea Assay Kit (BioAssay Systems, Destin, Fla.) (Schweitzer, F. et al., supra).
Histopathology and quantification of right ventricular and lung periarteriolar collagen deposition. 5 μm thick FFPE sections of the right heart and lung were cut and stained with Masson's trichrome and 0.1% Pico Sirius Red counterstained with Weigert's hematoxylin, to reveal fibrillar collagen, respectively by the UGA CVM Histopoathology Laboratory (Athens, Ga.). Whole slide images were acquired by Servicebio (Woburn, Mass.). To quantify right ventricular collagen deposition in Masson's trichrome stained sections, 170 mm² regions were analyzed using Image J software (imagej.nih.gov/ij), with the threshold color plugin set to RGB; bright blue collagen was selected, converted to a binary image, and measured to quantify the collagen area. Collagen deposition results are reported as:

$$\% RV collagen = \frac{total collagen area}{total muscle area} \times 100 \% .$$

To quantify periarteriolar collagen deposition in Picro-Sirius Red stained sections, the average of five small pulmonary vessels approximately <100 μm were analyzed using Image J software, with the color deconvolution plugin (www.mecourse.com/landinig/software/cdeconv/cdeconv.html) followed by application of the MRI fibrosis tool (dev.mri.cnrs.fr/projects/imagej-macros/wiki/Fibrosis Tool) to quantify percentage area of fibrosis using the default settings (red 1: 0148, green 1: 0772, blue 1: 0.618, red 2: 0.462, green 2: 0.602, blue 2: 0.651, red 3: 0.187, green 3: 0.523, blue 3: 0.831) (Arunsan, P. et al. Programmed knockout mutation of liver fluke granulin attenuates virulence of infection-induced hepatobiliary morbidity. Elife 8, doi:10.7554/eLife.41463 (2019)). Periarteriolar collagen deposition results are reported as:

$$\% Area threshold = \frac{total collagen area}{total vessel area} \times 100 \% .$$

Statistical analysis. All statistical analyses were performed using GraphPad Prism (GraphPad Software, La Jolla, Calif.). Serial and group characterizations of hemodynamics mPAP and RVSP were analyzed using repeated measures mixed modeling. Post hoc analysis was based on Fisher's least significant difference procedure for pairwise differences. Differences in cytokines, monocytes and macrophage phenotypes, and collagen deposition were analyzed using Mann-Whitney U tests. Difference in CD4⁺ T cells and viral load were analyzed by multiple analysis using the using the Holm-Sidak method, with α=0.05. To test for associations between mPAP and immune markers, Spearman correlation was performed as indicated by the Spearman R. All data represents the mean±SD.

### OTHER EMBODIMENTS

All publications, published patent applications, and patents mentioned in the above disclosure are herein incorporated by reference. Various modifications and variations of the described methods and compositions described and exemplified herein will be apparent to those skilled in the art without departing from the scope and spirit of the disclosure. Although the disclosure includes specific embodiments, it should be understood that the subject matter as claimed should not be unduly limited to such specific embodiments. Indeed, various modifications of the described modes for carrying out the embodiments as described and exemplified will be apparent to those skilled in molecular biology or related fields and are intended to be within the scope of the disclosed embodiments.

1. A composition for treating or preventing HIV-associated pulmonary arterial hypertension (HIV-PAH), the composition comprising an effective amount of one or more statins and one or more antiretroviral agents, wherein the composition treats or prevents HIV-PAH.

2. The composition of claim 1, wherein the one or more statins is selected from atorvastatin, fluvastatin, lovastatin, pravastatin, rosuvastatin, simvastatin, cerivastatin, pitavastatin, or a combination thereof.

3. The composition of claim 1, wherein the one or more antiretroviral agents comprises a nucleoside reverse transcriptase inhibitor, a non-nucleoside reverse transcriptase inhibitor, a protease inhibitor, a fusion or entry inhibitor, an integrase inhibitor, or a combination thereof.

4. (canceled)

5. The composition of claim 3, wherein the one or more antiretroviral agents comprises emtricitabine and tenofovir disoproxil fumarate.

6. The composition of claim 1, wherein the statin is atorvastatin and the one or more antiretroviral agents comprises emtricitabine and tenofovir disoproxil fumarate.

7-9. (canceled)

10. The composition of claim 1, wherein the composition reduces or prevents pulmonary vascular collagen deposition or right ventricular collagen deposition.

11. The composition of claim 1, wherein the composition prevents the onset of HIV-PAH.

12. (canceled)

13. A method of treating or preventing pulmonary arterial hypertension (PAH) in a subject infected with HIV or at risk of being infected by HIV, the method comprising administering to the subject an effective amount of:
a) one or more statins; and
b) one or more antiretroviral agents.

14. (canceled)

15. The method of claim 13, wherein the subject is at risk of being infected by HIV.

16-19. (canceled)

20. A method of treating or preventing pulmonary arterial hypertension (PAH) in a subject infected with HIV or at risk of being infected by HIV, the method comprising administering to the subject an effective amount of the composition of claim 1.

21. (canceled)

22. A method of preventing HIV-associated pulmonary arterial hypertension (HIV-PAH) in a subject diagnosed with HIV-PAH, the method comprising administering to a subject in need thereof an effective amount of the composition of claim 1 at the time that the subject is diagnosed with HIV.

23. The method of claim 22, wherein the method treats or prevents pulmonary vascular collagen deposition or right ventricular collagen deposition, or increases the levels of IL-15 in an HIV-infected subject or in a subject at risk of HIV infection, the method comprising administering to a subject in need thereof an effective amount of one or more statins and one or more antiretroviral agents.

24-31. (canceled)

32. The method of claim 13, wherein the subject is human.

33. The method of claim 32, wherein the subject is a human patient with HIV.

34. The method of claim 32, wherein the subject is a human patient and is treated at the time of diagnosis of HIV infection.

35. (canceled)

36. The method of claim 13, wherein one or more statins are administered prior to HIV infection or post-acute phase of infection.

37. A pharmaceutical pack comprising one or more statins and one or more antiretroviral agents, wherein the one or more statins and the one or more antiretroviral agents are formulated together or separately and in individual dosage amounts.

38. The pharmaceutical pack of claim 37, wherein the one or more statins and the one or more antiretroviral agents are formulated together.

39. The pharmaceutical pack of claim 37, wherein the one or more statins is selected from atorvastatin, fluvastatin, lovastatin, pravastatin, rosuvastatin, simvastatin, cerivastatin, pitavastatin, or a combination thereof.

40. The pharmaceutical pack of claim 37, wherein the one or more statins is atorvastatin.

41. The pharmaceutical pack of claim 37, wherein the one or more antiretroviral agents is a nucleoside reverse transcriptase inhibitor, a non-nucleoside reverse transcriptase inhibitor, a protease inhibitor, a fusion or entry inhibitor, an integrase inhibitor, or a combination thereof.

42. The pharmaceutical pack of claim 37, wherein the one or more antiretroviral agents comprises Zidovudine (Retrovir, AZT), Didanosine (Videx, Videx EC, ddI), Stavudine (Zerit, d4T), Lamivudine (Epivir, 3TC), Abacavir (Ziagen, ABC), Tenofovir, (Viread, TDF), Lamivudine, Combivir (combination of zidovudine and lamivudine), Trizivir (combination of zidovudine, lamivudine and abacavir), Emtricitabine (Emtriva, FTC), Truvada (combination of emtricitabine and Tenofovir (tenofovir disoproxil fumarate), Epzicom (combination of abacavir and lamivudine), Nevirapine (Viramune, NVP), Delavirdine (Rescriptor, DLV), Efavirenz (Sustiva or Stocrin, EFV, also part of Atripla), Etravirine (Intelence, ETR), Rilpivirine (Edurant, RPV, also part of Complera or Epivlera), Saquinavir (Invirase, SQV), Indinavir (Crixivan, IDV), Ritonavir (Norvir, RTV), Nelfinavir (Viracept, NFV), Amprenavir (Agenerase, APV), Lopinavir/ritonavir (Kaletra or Aluvia, LPV/RTV), Atazanavir (Reyataz, ATZ), Fosamprenavir (Lexiva, Telzir, FPV), Tipranavir (Aptivus, TPV), Darunavir (Prezista, DRV), Enfuvirtide (Fuzeon, ENF, T-20), Maraviroc (Selzentry or Celsentri, MVC), Raltegravir (Isentress, RAL), Elvitegravir (EVG, part of the combination Stribild), Dolutegravir (Tivicay, DTG), or combination thereof.

43. The pharmaceutical pack of claim 42, wherein the one or more antiretroviral agents comprises emtricitabine and tenofovir disoproxil fumarate.
